(19) United States
(12) Patent Application Publication
(10) Pub. No.: US 2025/0092029 A1
(43) Pub. Date: Mar. 20, 2025

(54) NOVEL AND HIGHLY SELECTIVE SARS-COV-2 MPRO INHIBITORS

(71) Applicant: THE WISTAR INSTITUTE OF ANATOMY AND BIOLOGY, Philadelphia, PA (US)

(72) Inventors: Joseph SALVINO, Chester Springs, PA (US); Luis MONTANER, Philadelphia, PA (US); Ian TIETJEN, Philadelphia, PA (US); Adi NARAYANA REDDY POLI, Philadelphia, PA (US)

(73) Assignee: THE WISTAR INSTITUTE OF ANATOMY AND BIOLOGY, Philadelphia, PA (US)

(21) Appl. No.: 18/727,389

(22) PCT Filed: Feb. 7, 2023
(86) PCT No.: PCT/US2023/062128

§ 371 Date: Jul. 9, 2024

Related U.S. Application Data
(60) Provisional application No. 63/307,558, filed on Feb. 7, 2022.

(51) Int. Cl. C07D 413/12; A61K 31/553; A61K 45/06; A61P 31/14

(52) U.S. Cl. CPC C07D 413/12; A61K 31/553; A61K 45/06; A61P 31/14

(57) ABSTRACT

Disclosed herein are compounds of the formulas (I) as well as analogs thereof, wherein the variables are defined herein. Also provided are pharmaceutical compositions thereof. In some aspects, the compounds and compositions provided herein may be used to inhibit Mpro proteases. Also provided are methods of administering compounds and compositions provided herein to a patient in need thereof, for example, for the treatment of diseases such as SARS-CoV-2 or a variant thereof.

This application claims the benefit of priority to U.S. Provisional Application No. 63/307,558, filed on Feb. 7, 2022, the entire contents of which are hereby incorporated by reference.

This work was made with government support under grant number CA010815 awarded by the National Institutes of Health. The government has certain rights in the invention.

### BACKGROUND

### I. Field

This disclosure relates to the fields of virology, biology, pharmacology, medicine, and chemistry. In particular, new compounds, compositions, and methods of treatment related to the treatment of SARS-CoV-2 are disclosed.

### II. Description of Related Art

Severe acute respiratory syndrome coronavirus 2 (SARS-CoV-2) has caused a global pandemic which continues to inflict substantial morbidity and mortality worldwide. As of October 2021, there were close to 250 million SAR-CoV-2 cases reported resulting in close to 4.8 million deaths worldwide. Although there are at least 21 distinct SARS-CoV-2 vaccines approved for emergency use globally (Zimmer et al., 2020), SARS-CoV-2 is rapidly evolving to generate variants of concern (VOC) with improved transmission and/or reduced responsiveness to current vaccine measures, particularly after partial vaccination (Lopez Bernal et al., 2021; Planas et al., 2021; Wang et al., 2021; Garcia-Beltran et al., 2021). Several VOC contain mutations in the SARS-CoV-2 spike receptor-binding domain (RDB) (Daniloski et al., 2021), the primary viral regulator of cell entry and main target of neutralizing antibody activity, and these mutations in turn drive impaired recognition of the virus by human antibody-mediated immunity (Wang et al., 2021; Zhou et al., 2021; Li et al., 2021). Furthermore, poor vaccine accessibility in many parts of the world, combined with vaccine hesitancy in vaccine-accessible regions, increase the risk of sustained SARS-CoV-2 infections and emergence of variants with vaccine breakthrough potential, demonstrating a necessity for additional viral countermeasures. Therefore, there remains a need to find new and unique compounds for the treatment of SARS-Cov-2.

### SUMMARY

In some aspects, the present disclosure provides novel compounds, including Mpro inhibitors, pharmaceutical compositions thereof, and methods for their use in the treatment of viral infections.

In some aspects, the present disclosure provides compounds of the formula:

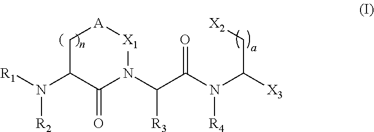

wherein:
A is O, S, or NR′, wherein R′ is hydrogen, alkyl_{(C≤8)}, or substituted alkyl_{(C≤8)};
X₁ is cycloalkanediyl_{(C≤12)}, arenediyl_{(C≤12)}, heteroarenediyl_{(C≤12)}, heterocycloalkanediyl_{(C≤12)}, or a substituted version of any of these groups;
X₂ is heteroarenediyl_{(C≤12)}, heterocycloalkanediyl_{(C≤12)}-R₆, or a substituted version thereof, or a group of the formula:

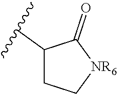

a is 0, 1, or 2;
X₃ is C(O)(CH₂)ₘR₅ or cyano;
m and n are each independently 0, 1, 2, or 3;
R₁ and R₂ are each independently selected from hydrogen, alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; or a monovalent amino protecting group; or R₁ and R₂ are a divalent amino protecting group; or Y₁—Rₐ; wherein:
Y₁ is —C(O)—, —C(O)O—, —C(O)NR_{b}—, —S(O)—, —S(O)ₓO—, or —S(O)ₓNR_{b}—; wherein:
x is 0, 1, or 2;
   R_{b} and R_{b′} are each independently hydrogen, alkyl_{(C≤12)}, substituted alkyl_{(C≤12)}, acyl_{(C≤12)}, substituted acyl_{(C≤12)}, or a monovalent amino protecting group; and
   Rₐ is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
R₃ is hydrogen, alkyl_{(C≤12)}, aryl_{(C≤12)}, aralkyl_{(C≤12)}, heteroaryl_{(C≤12)}, heterocycloalkyl_{(C≤12)}, or a substituted version of any of these groups; or is the side chain of one of the 20 canonical amino acids;
R₄ and R₆ are each independently selected from hydrogen, alkyl_{(C≤12)}, substituted alkyl_{(C≤12)}, acyl_{(C≤12)}, substituted acyl_{(C≤12)}, or a monovalent amino protecting group; and
R₅ is Y₂—R_{c}; wherein:
   Y₂ is —NR_{d}—, —S—, —O—, —C(O)—, —OC(O)—, —C(O)O—, —NR_{d}C(O)—, —C(O)NR_{d}—, —OC(O)O—, —OC(O)NR_{d}—, —NR_{d}C(O)O—, —NR_{d}C(O)NR_{d′}—, —S(O)_{y}—, —OS(O)_{y}—, —S(O)_{y}O—, —NR_{d}S(O)_{y}—, —S(O)_{y}NR_{d}—, —OS(O)_{y}O—, —OS(O)_{y}NR_{d}—, —NR_{d}S(O)_{y}O—, or —NR_{d}S(O)_{y}NR_{d′}—; wherein:
      y is 0, 1, or 2;
      R_{d} and R_{d′} are each independently hydrogen, alkyl_{(C≤12)}, substituted alkyl_{(C≤12)}, acyl_{(C≤12)}, substituted acyl_{(C≤12)}, or a monovalent amino protecting group; and
   R_{c} is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
      or a pharmaceutically acceptable salt thereof.

In some embodiments, the present disclosure provides compounds of the formula:

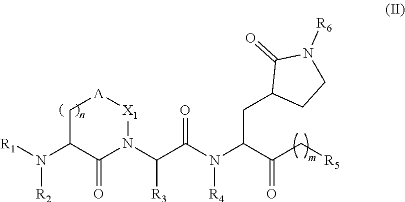

wherein:
A is O, S, or NR′, wherein R′ is hydrogen, alkyl_{(C≤8)}, or substituted alkyl_{(C≤8)};
X₁ is cycloalkanediyl_{(C≤12)}, arenediyl_{(C≤12)}, heteroarenediyl_{(C≤12)}, heterocycloalkanediyl_{(C≤12)}, or a substituted version of any of these groups;
m and n are each independently 0, 1, 2, or 3;
R₁ and R₂ are each independently selected from hydrogen, alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; or a monovalent amino protecting group; or R₁ and R₂ are a divalent amino protecting group; or Y₁—Rₐ; wherein:
   Y₁ is —C(O)—, —C(O)O—, —C(O)NR_{b}—, —S(O)ₓ—, —S(O)ₓO—, or —S(O)ₓNR_{b′}—; wherein:
      x is 0, 1, or 2;
      R_{b} and R_{b′} are each independently hydrogen, alkyl_{(C≤12)}, substituted alkyl_{(C≤12)}, acyl_{(C≤12)}, substituted acyl_{(C≤12)}, or a monovalent amino protecting group; and
   Rₐ is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
R₃ is hydrogen, alkyl_{(C≤12)}, aryl_{(C≤12)}, aralkyl_{(C≤12)}, heteroaryl_{(C≤12)}, heterocycloalkyl_{(C≤12)}, or a substituted version of any of these groups; or is the side chain of one of the 20 canonical amino acids;
R₄ and R₆ are each independently selected from hydrogen, alkyl_{(C≤12)}, substituted alkyl_{(C≤12)}, acyl_{(C≤12)}, substituted acyl_{(C≤12)}, or a monovalent amino protecting group; and
R₅ is Y₂—R_{c}; wherein:
   Y₂ is —NR_{d}—, —S—, —O—, —C(O)—, —OC(O)—, —C(O)O—, —NR_{d}C(O)—, —C(O)NR_{d}—, —OC(O)O—, —OC(O)NR_{d}—, —NR_{d}C(O)O—, —NR_{d}C(O)NR_{d′}—, —S(O)_{y}—, —OS(O)_{y}—, —S(O)_{y}O—, —NR_{d}S(O)_{y}—, —S(O)_{y}NR_{d}—, —OS(O)_{y}O—, —OS(O)_{y}NR_{d}—, —NR_{d}S(O)_{y}O—, or —NR_{d}S(O)_{y}NR_{d′}—; wherein:
      y is 0, 1, or 2;
      R_{d} and R_{d′} are each independently hydrogen, alkyl_{(C≤12)}, substituted alkyl_{(C≤12)}, acyl_{(C≤12)}, substituted acyl_{(C≤12)}, or a monovalent amino protecting group; and
   R_{c} is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
      or a pharmaceutically acceptable salt thereof.

In some embodiments, the present disclosure provides compounds of the formula:

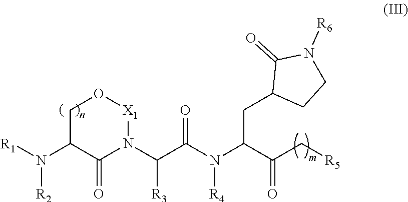

wherein:
X₁ is cycloalkanediyl_{(C≤12)}, arenediyl_{(C≤12)}, heteroarenediyl_{(C≤12)}, heterocycloalkanediyl_{(C≤12)}, or a substituted version of any of these groups;
m and n are each independently 0, 1, 2, or 3;
R₁ and R₂ are each independently selected from hydrogen, alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; or a monovalent amino protecting group; or R₁ and R₂ are a divalent amino protecting group; or Y₁—Rₐ; wherein:
   Y₁ is —C(O)—, —C(O)O—, —C(O)NR_{b}—, —S(O)ₓ—, —S(O)ₓO—, or —S(O)ₓNR_{b′}—; wherein:
      x is 0, 1, or 2;
      R_{b} and R_{b′} are each independently hydrogen, alkyl_{(C≤12)}, substituted alkyl_{(C≤12)}, acyl_{(C≤12)}, substituted acyl_{(C≤12)}, or a monovalent amino protecting group; and
   Rₐ is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
R₃ is hydrogen, alkyl_{(C≤12)}, aryl_{(C≤12)}, aralkyl_{(C≤12)}, heteroaryl_{(C≤12)}, heterocycloalkyl_{(C≤12)}, or a substituted version of any of these groups; or is the side chain of one of the 20 canonical amino acids;
R₄ and R₆ are each independently selected from hydrogen, alkyl_{(C≤12)}, substituted alkyl_{(C≤12)}, acyl_{(C≤12)}, substituted acyl_{(C≤12)}, or a monovalent amino protecting group; and
R₅ is Y₂—R_{c}; wherein:
   Y₂ is —NR_{d}—, —S—, —O—, —C(O)—, —OC(O)—, —C(O)O—, —NR_{d}C(O)—, —C(O)NR_{d}—, —OC(O)O—, —OC(O)NR_{d}—, —NR_{d}C(O)O—, —NR_{d}C(O)NR_{d′}—, —S(O)_{y}—, —OS(O)—, —S(O)_{y}O—, —NR_{b}S(O)_{y}—, —S(O)_{y}NR_{d}—, —OS(O)O—, —OS(O)_{y}NR_{d}—, —NR_{d}S(O)O—, or —NR_{d}S(O)_{y}NR_{d′}—; wherein:
      y is 0, 1, or 2;
      R_{d} and R_{d′} are each independently hydrogen, alkyl_{(C≤12)}, substituted alkyl_{(C≤12)}, acyl_{(C≤12)}, substituted acyl_{(C≤12)}, or a monovalent amino protecting group; and
   R_{c} is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
      or a pharmaceutically acceptable salt thereof.

In some embodiments, the present disclosure provides compounds of the formula:

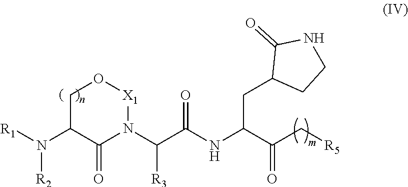

wherein:
X₁ is cycloalkanediyl_{(C≤12)}, arenediyl_{(C≤12)}, heteroarenediyl_{(C≤12)}, heterocycloalkanediyl_{(C≤12)}, or a substituted version of any of these groups;
m and n are each independently 0, 1, 2, or 3;
R₁ and R₂ are each independently selected from hydrogen, alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; or a monovalent amino protecting group; or R₁ and R₂ are a divalent amino protecting group; or Y₁—Rₐ; wherein:
   Y₁ is —C(O)—, —C(O)O—, —C(O)NR_{b}—, —S(O)ₓ—, —S(O)ₓO—, or —S(O)ₓNR_{b′}—; wherein:
      x is 0, 1, or 2;
      R_{b} and R_{b′} are each independently hydrogen, alkyl_{(C≤12)}, substituted alkyl_{(C≤12)}, acyl_{(C≤12)}, substituted acyl_{(C≤12)}, or a monovalent amino protecting group; and
   Rₐ is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
R₃ is hydrogen, alkyl_{(C≤12)}, aryl_{(C≤12)}, aralkyl_{(C≤12)}, heteroaryl_{(C≤12)}, heterocycloalkyl_{(C≤12)}, or a substituted version of any of these groups; or is the side chain of one of the 20 canonical amino acids; and
R₅ is Y₂—R_{c}; wherein:
   Y₂ is —NR_{d}—, —S—, —O—, —C(O)—, —OC(O)—, —C(O)O—, —NR_{d}C(O)—, —C(O)NR_{d}—, —OC(O)O—, —OC(O)NR_{d}—, —NR_{d}C(O)O—, —NR_{d}C(O)NR_{d′}—, —S(O)_{y}—, —OS(O)—, —S(O)_{y}O—, —NR_{d}S(O)_{y}—, —S(O)_{y}NR_{d}—, —OS(O)_{y}O—, —OS(O)_{y}NR_{d}—, —NR_{d}S(O)_{y}O—, or —NR_{d}S(O)_{y}NR_{d′}—; wherein:
      y is 0, 1, or 2;
      R_{d} and R_{d}, are each independently hydrogen, alkyl_{(C≤12)}, substituted alkyl_{(C≤12)}, acyl_{(C≤12)}, substituted acyl_{(C≤12)}, or a monovalent amino protecting group; and
   R_{c} is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
or a pharmaceutically acceptable salt thereof.

In some embodiments, the present disclosure provides compounds of the formula:

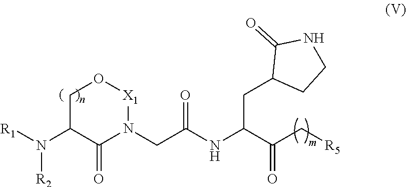

wherein:
X₁ is cycloalkanediyl_{(C≤12)}, arenediyl_{(C≤12)}, heteroarenediyl_{(C≤12)}, heterocycloalkanediyl_{(C≤12)}, or a substituted version of any of these groups;
m and n are each independently 0, 1, 2, or 3;
R₁ and R₂ are each independently selected from hydrogen, alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; or a monovalent amino protecting group; or R₁ and R₂ are a divalent amino protecting group; or Y₁—Rₐ; wherein:
   Y₁ is —C(O)—, —C(O)O—, —C(O)NR_{b}—, —S(O)ₓ—, —S(O)ₓO—, or —S(O)ₓNR_{b′}—; wherein:
      x is 0, 1, or 2;
      R_{b} and R_{b′} are each independently hydrogen, alkyl_{(C≤12)}, substituted alkyl_{(C≤12)}, acyl_{(C≤12)}, substituted acyl_{(C≤12)}, or a monovalent amino protecting group; and
   Rₐ is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; and
R₅ is Y₂—R_{c}; wherein:
   Y₂ is —NR_{d}—, —S—, —O—, —C(O)—, —OC(O)—, —C(O)O—, —NR_{d}C(O)—, —C(O)NR_{d}—, —OC(O)O—, —OC(O)NR_{d}—, —NR_{d}C(O)O—, —NR_{d}C(O)NR_{d}—, —S(O)_{y}—, —OS(O)—, —S(O)_{y}O—, —NR_{d}S(O)_{y}—, —S(O)NR_{d}—, —OS(O)_{y}O—, —OS(O)_{y}NR_{d}—, —NR_{d}S(O)_{y}O—, or —NR_{d}S(O)_{y}NR_{d′}—; wherein:
      y is 0, 1, or 2;
      R_{d} and R_{d′} are each independently hydrogen, alkyl_{(C≤12)}, substituted alkyl_{(C≤12)}, acyl_{(C≤12)}, substituted acyl_{(C≤12)}, or a monovalent amino protecting group; and
   R_{c} is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
      or a pharmaceutically acceptable salt thereof.

In some embodiments, the present disclosure provides compounds of the formula:

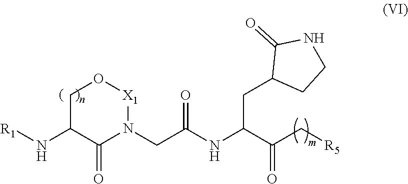

wherein:
X₁ is cycloalkanediyl_{(C≤12)}, arenediyl_{(C≤12)}, heteroarenediyl_{(C≤12)}, heterocycloalkanediyl_{(C≤12)}, or a substituted version of any of these groups;
m or n are each independently 0, 1, 2, or 3;
R₁ is hydrogen, alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; or a monovalent amino protecting group; or Y₁—Rₐ; wherein:
   Y₁ is —C(O)—, —C(O)O—, —C(O)NR_{b}—, —S(O)ₓ—, —S(O)ₓO—, or —S(O)ₓNR_{b′}—; wherein:
      x is 0, 1, or 2;
      R_{b} and R_{b′} are each independently hydrogen, alkyl_{(C≤12)}, substituted alkyl_{(C≤12)}, acyl_{(C≤12)}, substituted acyl_{(C≤12)}, or a monovalent amino protecting group; and
   Rₐ is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; and
R₅ is Y₂—R_{c}; wherein:
   Y₂ is —NR_{d}—, —S—, —O—, —C(O)—, —OC(O)—, —C(O)O—, —NR_{d}C(O)—, —C(O)NR_{d}—, —OC(O)O—, —OC(O)NR_{d}—, —NR_{d}C(O)O—, —NR_{d}C(O)NR_{d′}—, —S(O)_{y}—, —OS(O)_{y}—, —S(O)O—, —NR_{d}S(O)_{y}—, —S(O)ₓNR—, —OS(O)_{y}O—, —OS(O)_{y}NR_{d}—, —NR_{d}S(O)_{y}O—, or —NR_{d}S(O)_{y}NR_{d′}—; wherein:
      y is 0, 1, or 2;
      R_{d} and R_{d′} are each independently hydrogen, alkyl_{(C≤12)}, substituted alkyl_{(C≤12)}, acyl_{(C≤12)}, substituted acyl_{(C≤12)}, or a monovalent amino protecting group; and
   R_{c} is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
      or a pharmaceutically acceptable salt thereof.

In some embodiments, the present disclosure provides compounds of the formula:

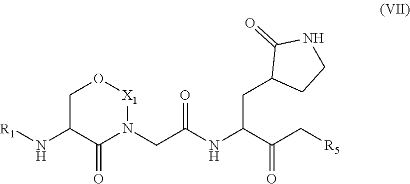

wherein:
X₁ is cycloalkanediyl_{(C≤12)}, arenediyl_{(C≤12)}, heteroarenediyl_{(C≤12)}, heterocycloalkanediyl_{(C≤12)}, or a substituted version of any of these groups;
R₁ is hydrogen, alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; or a monovalent amino protecting group; or Y₁—Rₐ; wherein:
   Y₁ is —C(O)—, —C(O)O—, —C(O)NR_{b}—, —S(O)ₓ—, —S(O)ₓO—, or —S(O)ₓNR_{b′}—; wherein:
      x is 0, 1, or 2;
      R_{b} and R_{b′} are each independently hydrogen, alkyl_{(C≤12)}, substituted alkyl_{(C≤12)}, acyl_{(C≤12)}, substituted acyl_{(C≤12)}, or a monovalent amino protecting group; and
   Rₐ is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; and
R₅ is Y₂—R_{c}; wherein:
   Y₂ is —NR_{d}—, —S—, —O—, —C(O)—, —OC(O)—, —C(O)O—, —NR_{d}C(O)—, —C(O)NR_{d}—, —OC(O)O—, —OC(O)NR_{d}—, —NR_{d}C(O)O—, —NR_{d}C(O)NR_{d′}—, —S(O)_{y}—, —OS(O)_{y}—, —S(O)_{y}O—, —NR_{d}S(O)_{y}—, —S(O)NR_{d}—, —OS(O)_{y}O—, —OS(O)_{y}NR_{d}—, —NR_{d}S(O)_{y}O—, or —NR_{d}S(O)_{y}NR_{d′}—; wherein:
      y is 0, 1, or 2;
      R_{d} and R_{d′} are each independently hydrogen, alkyl_{(C≤12)}, substituted alkyl_{(C≤12)}, acyl_{(C≤12)}, substituted acyl_{(C≤12)}, or a monovalent amino protecting group; and
   R_{c} is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
      or a pharmaceutically acceptable salt thereof.

In some embodiments, X₁ is arenediyl_{(C≤12)} or substituted arenediyl_{(C≤12)}. In further embodiments, X₁ is arenediyl_{(C≤12)}, such as benzenediyl. In some embodiments, R₁ is Y₁—Rₐ. In further embodiments, Y₁ is —C(O)—, —C(O)O—, or —C(O)NR_{b}—. In some embodiments, Y₁ is —C(O)O—. In other embodiments, Y₁ is —C(O)—. In some embodiments, Rₐ is aralkyl_{(C≤12)} or substituted aralkyl_{(C≤12)}. In some embodiments, Rₐ is aralkyl_{(C≤12)}, such as benzyl. In other embodiments, Rₐ is aryl_{(C≤12)} or substituted aryl_{(C≤12)}. In some embodiments, Rₐ is aryl_{(C≤12)}, such as phenyl. In still other embodiments, Rₐ is heteroaryl_{(C≤12)} or substituted heteroaryl_{(C≤12)}. In some embodiments, Rₐ is heteroaryl_{(C≤12)}, such as quinolyl or 2-quinolyl.

In some embodiments, Y₂ is —OC(O)— or —C(O)O—. In some embodiments Y₂ is —OC(O)—. In some embodiments, R_{c} is aryl_{(C≤12)} or substituted aryl_{(C≤12)}. In some embodiments, R_{c} is substituted aryl_{(C≤12)}. In further embodiments, R_{c} is haloaryl_{(C≤12)}, such as 2,6-dichlorophenyl.

In some embodiments, m is 0, 1, or 2. In further embodiments, m is 0 or 1. In other embodiments, m is 1 or 2. In some embodiments, m is 1. In some embodiments, n is 0, 1, or 2. In further embodiments, n is 0 or 1. In other embodiments, n is 1 or 2. In some embodiments, n is 1.

In some embodiments, R₂ is hydrogen. In some embodiments, R₃ is hydrogen. In some embodiments, R₄ is hydrogen. In some embodiments, R₆ is hydrogen.

In some embodiments, the compounds are further defined as:

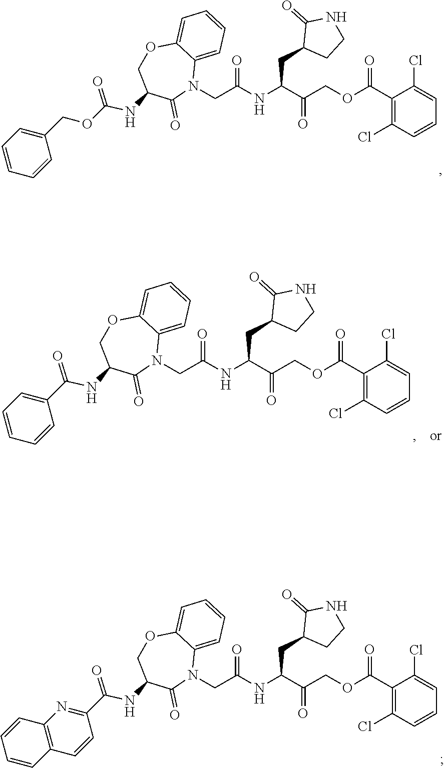 [SMILES: O=C(CN1C(=O)[C@@H](NC(=O)OCc2ccccc2)COc2ccccc21)N[C@@H](C[C@@H]1CCNC1=O)C(=O)COC(=O)c1c(Cl)cccc1Cl ; O=C(CN1C(=O)[C@@H](NC(=O)c2ccccc2)COc2ccccc21)N[C@@H](C[C@@H]1CCNC1=O)C(=O)COC(=O)c1c(Cl)cccc1Cl ; O=C(CN1C(=O)[C@@H](NC(=O)c2ccc3ccccc3n2)COc2ccccc21)N[C@@H](C[C@@H]1CCNC1=O)C(=O)COC(=O)c1c(Cl)cccc1Cl]

or a pharmaceutically acceptable salt thereof.

In another aspect, the present disclosure provides pharmaceutical compositions comprising:
(A) a compound described herein; and
(B) an excipient.

In some embodiments, the pharmaceutical compositions are formulated for administration systemically. In some embodiments, the pharmaceutical compositions are formulated as a unit dose.

In another aspect, the present disclosure provides methods of treating a disease or disorder in a patient comprising administering to the patient in need thereof a therapeutically effective dose of a compound or pharmaceutical composition described herein. In some embodiments, the disease or disorder is a viral infection. In further embodiments, the viral infection is the infection of a coronavirus, such as SARS-CoV-2 or a variant thereof. In some embodiments, the patient is a mammal, such as a human. In some embodiments, the patient has been diagnosed with the infection. In other embodiments, the patient has not been diagnosed with the infection.

In some embodiments, the compound is administered with a second therapeutic agent, such as molnupiravir, paxlovid, or remdesivir. In some embodiments, the second therapeutic agent is remdesivir. In some embodiments, the methods comprise administering less than a therapeutically effective dose of remdesivir. In some embodiments, the methods comprise administering less than a therapeutically effective dose of the compound when the compound is administered alone. In further embodiments, the methods comprise administering both remdesivir and the compound in less than a therapeutically effective dose. In some embodiments, the compound is administered for 1 day to 20 days. In further embodiments, the compound is administered for 3 days to 5 days. In some embodiments, the compound is administered once. In other embodiments, the compound is administered two or more times.

It is contemplated that any method or composition described herein can be implemented with respect to any other method or composition described herein. For example, a compound synthesized by one method may be used in the preparation of a final compound according to a different method.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-B—Effects of compounds on CPE in Vero-E6 cells following SARS-CoV-2 infection. A, Representative brightfield images of uninfected and SARS-CoV-2 USA-WA1/2020 variant-infected Vero-E6 cells following 4 days' incubation in the absence or presence of representative compounds. Arrows denote examples of CPE. Scale bars=100 μm. B, Dose-response curves of Mpro inhibitors and remdesivir on viral replication in Vero-E6 cells after 4 days' infection. Data are presented relative to cells treated with 0.1% DMSO vehicle control.

FIGS. 2A-C—Effects of compounds on CPE in Vero-E6 cells infected with SARS-CoV-2 VOC. A, Representative brightfield images of Vero-E6 cells infected with B.1.1.7 or B.1.351 VOC following 4 days' incubation in the absence or presence of representative compounds. Arrows denote examples of CPE. Scale bars=100 μm. B—C, Dose-response curves of GC-376 (B) and AP-8-013 (C) on viral replication in Vero-E6 cells after 4 days' infection. Data are presented relative to cells treated with 0.1% DMSO vehicle control.

FIG. 3—Effects of control inhibitors GC-376 and PF07321332 and AP-8-013 on SARS-CoV-2 replication in VeroE6 cells.

FIG. 4—Effects of control inhibitors GC-376, PF07321332, and AP-8-013 on SARS-CoV-2 replication.

FIG. 5—Effects of AP-8-013 in combination with remdesivir in Vero-E6 cells following 4 days' infection with SARS-CoV-2 (USA-WA1/2020 variant). Treatment with 3 or 5 μM AP-8-013 plus 1 or 3 μM remdesivir reduce the percent of wells with CPE relative to cells treated with AP-8-013 alone (dotted line) or remdesivir alone (left-most values). Data are presented relative to cells treated with 0.1% DMSO vehicle control.

FIG. 6—AP-8-013 is effective against the wild type, beta, and delta Covid 19 coronavirus variants of concern in reducing viral load. Briefly, Vero-E6 cells were treated with compounds before infection with 50× TCID50 of WT (USA-WA1/2020), Beta (B.1.351) or Delta (B.1.617.2) virus. After 48 h, cells were fixed, immunostained for viral nucleocapsid, treated with Hoechst dye to detect cell nuclei, and counted for infected and total cells by high-content imaging. Results indicate percent infected live cells.

FIG. 7—Mean plasma and lung concentration-time profiles of AP-8-013 after single IP dose at 10 mg/kg in male Hamster (N=3/timepoint).

### DESCRIPTION OF ILLUSTRATIVE EMBODIMENTS

Provided herein are compounds and compositions that may be used to inhibit a viral main protease (Mpro) and are thus useful in the treatment in a variety of coronaviruses. In some embodiments, these compounds may be used to treat SARS-CoV-2. In particular, these compounds may be used independently or may be used in combination with a polymerase inhibitor, such as remdesivir.

### I. Compounds and Formulations Thereof

### A. Compounds

The compounds of the present disclosure are shown, for example, above, in the summary of the invention section, the Examples section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All the compounds of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all compounds of the present disclosure are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present disclosure have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the art, whether for use in the indications stated herein or otherwise.

The compounds of the present disclosure may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration. In some embodiments, the present compounds may contain two or more atoms which have a defined stereochemical orientation.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present disclosure function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the disclosure contemplates prodrugs of the compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

### B. Formulations

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB. J., 22(3):659-661, 2008, which is incorporated herein by reference):

HED (mg/kg)=Animal dose (mg/kg)×(Animal Kₘ/Human Kₘ)

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### II. Methods of Treatment and Combination Therapies

### A. Methods of Treatment

In particular, the compositions that may be used in treating a disease or disorder in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., slowing, stopping, reducing or eliminating one or more symptoms or underlying causes of disease). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms and other drugs being administered concurrently. In some embodiments, the amount of the compounds used is calculated to be from about 0.01 mg to about 10,000 mg/day. In some embodiments, the amount is from about 1 mg to about 1,000 mg/day. In some embodiments, the compounds may be administered for 1 day to 20 days. In further embodiments, it is contemplated that the compounds may be administered for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days, or any range derivable therein. In some embodiments, the compounds may be administered for between 3 and 5 days, inclusive. In some embodiments, the compounds may be administered once. It is also contemplated that in some embodiments, the compounds disclosed herein may be administered two or more times. In some embodiments, these dosings may be reduced or increased based upon the biological factors of a particular patient such as increased or decreased metabolic breakdown of the drug or decreased uptake by the digestive tract if administered orally. Additionally, the compounds may be more efficacious and thus a smaller dose is required to achieve a similar effect. Such a dose is typically administered once a day for a few weeks or until sufficient achieve clinical benefit.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, family history, and the like).

### B. Combination Therapies

It is envisioned that the compounds described herein may be used in combination therapies with one or more additional therapies or a compound which mitigates one or more of the side effects experienced by the patient. It is common in the field of medicine to combine therapeutic modalities. The following is a general discussion of therapies that may be used in conjunction with the therapies of the present disclosure.

To treat diseases or disorders using the methods and compositions of the present disclosure, one would generally contact a cell or a subject with a compound and at least one other therapy. These therapies would be provided in a combined amount effective to achieve a reduction in one or more disease parameter. This process may involve contacting the cells/subjects with both agents/therapies at the same time, e.g., using a single composition or pharmacological formulation that includes both agents, or by contacting the cell/subject with two distinct compositions or formulations, at the same time, wherein one composition includes the compound and the other includes the other agent. In some embodiments, the compounds of the present disclosure or any therapies used in conjunction with the compounds of the present disclosure may be administered in a less than therapeutically effective dose when used either alone or in combination.

Alternatively, the compounds described herein may precede or follow the other treatment by intervals ranging from minutes to months. Non-limiting examples of such combination therapy include combination of one or more compounds of the invention with another antiviral agent, an anti-inflammatory agent, an immunosuppressant agent, a chemotherapeutic agent, radiation therapy, an antidepressant, an antipsychotic agent, an anticonvulsant, a neutralizing antibody, a mood stabilizer, an anti-infective agent, an antihypertensive agent, a cholesterol-lowering agent or other modulator of blood lipids, an agent for promoting weight loss, an antithrombotic agent, an agent for treating or preventing cardiovascular events such as myocardial infarction or stroke, an antidiabetic agent, an agent for reducing transplant rejection or graft-versus-host disease, an anti-arthritic agent, an analgesic agent, an anti-asthmatic agent or other treatment for respiratory diseases, or an agent for treatment or prevention of skin disorders. Compounds of the invention may be combined with agents designed to improve a patient's immune response to cancer, including (but not limited to) cancer vaccines. See Lu et al. (2011), which is incorporated herein by reference.

It also is conceivable that more than one administration of either the compound or the other therapy will be desired. Various combinations may be employed, where a compound of the present disclosure is “A,” and the other therapy is “B,” as exemplified below:
A/B/A B/A/B B/B/A A/A/B B/A/A A/B/B B/B/B/A B/B/A/B A/A/B/B A/B/A/B A/B/B/A B/B/A/A B/A/B/A B/A/A/B B/B/B/A A/A/A/B B/A/A/A A/B/A/A A/A/B/A A/B/B/B B/A/B/B B/B/A/B
   Other combinations are also contemplated.

### III. SARS-COV-2

SARS-CoV-2 contains two overlapping open reading frames at the end of the 5′ terminal which encode for two essential polypeptides called pp1a and pp1ab. These polypeptides produce most of the proteins involved in the replicase-transcriptase complex, the large majority of which are processed by the chymotrypsin-like cysteine protease (3CLpro or Mpro) (Thiel et al., 2003; Fan et al., 2004). at ≥11 viral cleavage sites. As these cleavage events result in release of mature non-structural proteins (Nspl-16) required for further viral replication and transmission (Thiel et al., 2003), loss of Mpro activity by therapeutic targeting would be expected to block progression of SARS-CoV-2 replication. In some embodiments, the compounds of the present disclosure inhibit Mpro. The active sites of Mpro are highly conserved across coronaviruses (Zhang et al., 2020), Mpro inhibitors may impart a higher genetic barrier to evolving SARS-CoV-2 drug resistance when these Mpro inhibitors are administrated either alone or in combination with agents that target other aspects of viral replication. In some embodiments, the compounds of the present disclosure may therefore be administered alone or in combination with at least one other antiviral agent. In some embodiments, the antiviral agents include molnupiravir, paxlovid, or remdesivir.

The present disclosure additionally contemplates treatment of other coronaviruses. In some embodiments, compounds of the present disclosure may be useful against SARS coronavirus (SARS-CoV), which causes severe acute respiratory syndrome (SARS). The compounds of the present disclosure may therefore be used to treat SARS. In some embodiments, compounds of the present disclosure may be useful against MERS coronavirus (MERS-CoV), which causes Middle East Respiratory Syndrome (MERS). The compounds of the present disclosure may therefore be used to treat MERS. The compounds of the present disclosure may also, in some embodiments, be used to treat any one of the common human coronaviruses 229E, NL63, OC43, or HKU1. In some embodiments, the present disclosure describes methods for treating patients who have been diagnosed with a viral infection. In some embodiments the diagnosed viral infection is the infection of a coronavirus, particularly a SARS-CoV-2 infection. In particular, the compounds may be used to treat one or more of the variants of SARS-CoV-2, including infection of the alpha, beta, delta, or omicron variants. In certain embodiments, compounds of the present disclosure may be used to treat a patient who has not been diagnosed with an infection. In particulate, the patient may not have been diagnosed with SARS-CoV-2. In other aspects, the patient may have been diagnosed with SARS-CoV-2 through an OTC test. In other aspects, the patient may have been diagnosed with SARS-CoV-2 through a PCR test. In other aspects, the patient is experiencing one or more symptoms of the disease.

### IV. Chemistry Background

In some aspects, compounds of this disclosure can be synthesized using the methods of organic chemistry as described in this application. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

The synthetic methods described herein can be further modified and optimized for preparative, pilot- or large-scale production, either batch of continuous, using the principles and techniques of process chemistry as applied by a person skilled in the art. Such principles and techniques are taught, for example, in Practical Process Research & Development (2000), which is incorporated by reference herein. The synthetic method described herein may be used to produce preparative scale amounts of the compounds described herein.

### A. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means=NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O—or a deprotonated form thereof; “mercapto” means —SH; and “thio” means=S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “----” represents an optional bond, which if present is either single or double. The symbol “-̅-̅-̅-̅” represents a single bond or a double bond. Thus, the formula

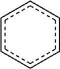

cover for example,

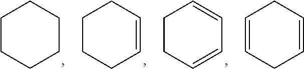

and

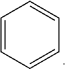

And it is understood that no one such ring atom forms part of more than one double bond. Furthermore, it is noted that the covalent bond symbol “-”, when connecting one or two stereogenic atoms, does not indicate any preferred stereochemistry. Instead, it covers all stereoisomers as well as mixtures thereof. The symbol “ [symbol] ”, when drawn perpendicularly across a bond

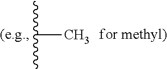

indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

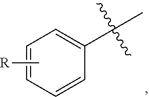

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

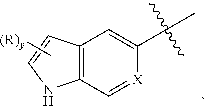

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C1-4-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino_{(C6)} group. Likewise, phenylethyl is an example of an aralkyl_{(C-8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic n system. An aromatic compound or chemical group may be depicted as a single resonance structure; however, depiction of one resonance structure is taken to also refer to any other resonance structure. For example:

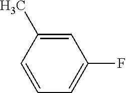

is also taken to refer to

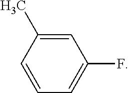

Aromatic compounds may also be depicted using a circle to represent the delocalized nature of the electrons in the fully conjugated cyclic n system, two non-limiting examples of which are shown below:

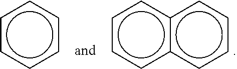

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂—are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

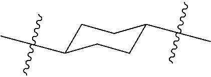 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂—are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

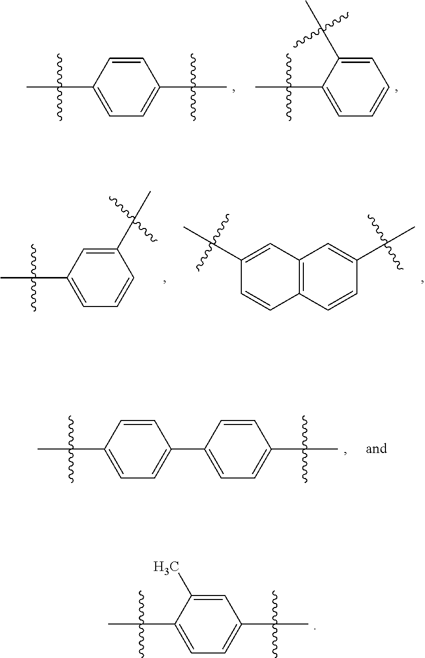

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heteroaralkyl” refers to the monovalent group -alkanediyl-heteroaryl, in which the terms alkanediyl and heteroaryl are each used in a manner consistent with the definitions provided above. Non-limiting examples are: pyridinylmethyl and 2-quinolinyl-ethyl.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “heterocycloalkalkyl” refers to the monovalent group -alkanediyl-heterocycloalkyl, in which the terms alkanediyl and heterocycloalkyl are each used in a manner consistent with the definitions provided above. Non-limiting examples are: morpholinylmethyl and piperidinylethyl.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C6H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. The term “haloaryl” is a subset of substituted aryl, in which the hydrogen atom replacement is limited to halo (i.e. —F, —Cl, —Br, or —I) such that no other atoms aside from carbon, hydrogen and halogen are present. The group, —C₆H₄Cl is a non-limiting example of a haloaryl group. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients. Unless otherwise noted, the term “about” is used to indicate a value of ±10% of the reported value, preferably a value of ±5% of the reported value. It is to be understood that, whenever the term “about” is used, a specific reference to the exact numerical value indicated is also included.”

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

An “amine protecting group” or “amino protecting group” is well understood in the art. An amine protecting group is a group which modulates the reactivity of the amine group during a reaction which modifies some other portion of the molecule. Amine protecting groups can be found at least in Greene and Wuts, 1999, which is incorporated herein by reference. Some non-limiting examples of amino protecting groups include formyl, acetyl, propionyl, pivaloyl, t-butylacetyl, 2-chloroacetyl, 2-bromoacetyl, trifluoroacetyl, trichloroacetyl, 0-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; alkylaminocarbonyl groups (which form ureas with the protect amine) such as ethylaminocarbonyl and the like; aralkyl groups such as benzyl, triphenylmethyl, benzyloxymethyl and the like; and silyl groups such as trimethylsilyl and the like. Additionally, the “amine protecting group” can be a divalent protecting group such that both hydrogen atoms on a primary amine are replaced with a single protecting group. In such a situation the amine protecting group can be phthalimide (phth) or a substituted derivative thereof wherein the term “substituted” is as defined above. In some embodiments, the halogenated phthalimide derivative may be tetrachlorophthalimide (TCphth). When used herein, a “protected amino group”, is a group of the formula PG_{MA}NH— or PG_{DA}N— wherein PG_{MA} is a monovalent amine protecting group, which may also be described as a “monovalently protected amino group” and PG_{DA} is a divalent amine protecting group as described above, which may also be described as a “divalently protected amino group”.

The term “canonical amino acid” refers to any one of alanine, arginine, asparagine, aspartate (or aspartic acid), cysteine, glutamine, glutamate (or glutamic acid), glycine, histidine, isoleucine, leucine, lysine, methionine, phenylalanine, proline, serien, threonine, tryptophan, tyrosine, or valine. The “side chain” of the amino acid refers to any atom or group(s) of atoms attached to the α-carbon of the amino acid molecule.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half. The term “EC₅₀” refers to an amount that is an effective concentration to results in a half-maximal response.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity with in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### V. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Design and Synthesis of Mpro Inhibitors

A highly selective Mpro inhibitor which only targets the viral protein versus inhibiting mammalian proteases involved in normal protein processing is expected to avoid significant toxicity during clinical development. A benzoxazepine acetic acid (Dolle et al. 1997; Itoh et al., 1986a; Itoh et al., 1986b) constrained P3-P2 mimetic previously used to synthesize conformationally restricted angiotensin converting enzyme (ACE) and ICE inhibitors was prioritized for synthesis since this appeared to closely mimic the bioactive conformation of peptide or inhibitor bound Mpro inhibitors (Wang et al., 2020).

A prototype highly constrained and selective peptidomimetic Mpro inhibitor, AP-8-013, was synthesized using the following method shown in Scheme 1. The nucleophilic oxygen atom of N-Boc L-serine reacted with 2-fluoro-nitrobenzene under base catalyzed conditions to form the aryl ether, A, which was then treated using Pd catalyzed reduction conditions in the presence of H₂ gas to reduce the aryl nitro group to the primary amine, B. Compound B was then reacted under T3P amide bond coupling-cyclization conditions to provide the P3-mimetic, C.

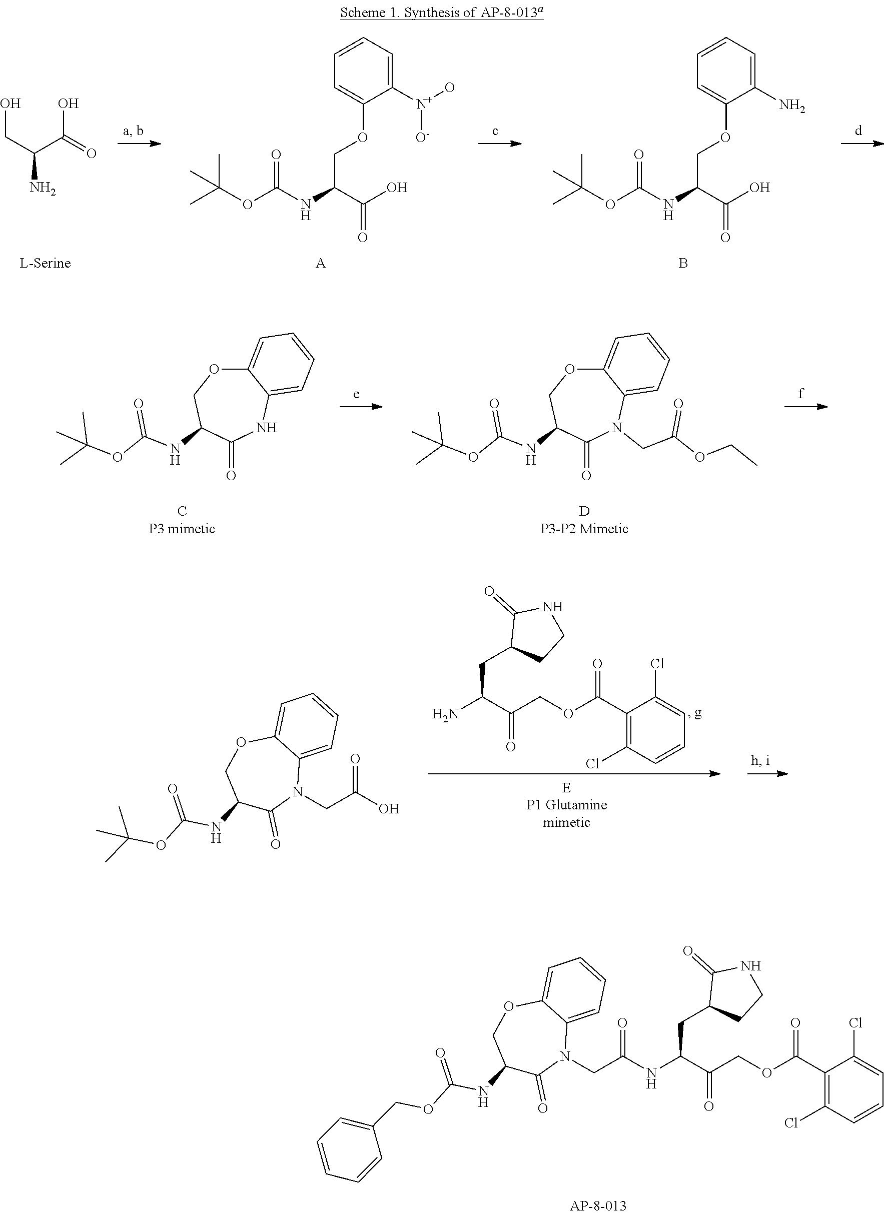

^{a}Reagents and conditions: (a) (BOC)₂O, aq NaHCO₃, Dioxane, 0° C.—rt, 2 h. (b) NaH, DMF, 1-fluoro-2-nitrobenzene, 0° C.-40 C, 2 h. (c) 10% Pd/C, H_{2(g)}, EtOH, rt, 16 h. (d) 50% T₃P in CH₂Cl₂, −20° C.-0° C., 1 h. (e) LiHMDS, THF, ethyl 2-bromoacetate, −78° C.—rt, 12 h. (f) NaOH, THF:MeOH:H₂O, 0° C.—rt, 12 h. (g) EDCI, Et₃N, DMF, (S)-3-amino-2-oxo-4-((S)-2-oxopyrrolidin-3-yl)butyl 2,6-dichloro benzoate. (h) 20% TFA in CH₂Cl₂, 0° C.—rt, 2 h (i) Cbz chloride, Et₃N, CH₂Cl₂, 0° C.—rt, 24 h.

Compound C was then treated with the strong base, LHMDS, followed by ethyl bromo-acetate under N-alkylation conditions to provide the N-Boc protected P3-P2-mimetic as the ethyl ester, D. Compound D was then treated under aqueous basic conditions to hydrolyze the ethyl ester to form the free carboxylic acid that was reacted with the acyoxymethyl ketone P1 glutamine mimetic, E, in the presence of the amide bond coupling reagent EDCI. The coupled product was reacted under acidic conditions to remove the N-Boc protecting group to provide the primary amine, which was reacted with Cbz-chloride in the presence of an organic base to form the N-Cbz to provide the fully elaborated peptide mimetic AP-8-013 (Scheme 1).

The Pfizer reference compound (Compound 22, analogous to PF-00835231) (Hoffman et al., 2020) and termed herein AP-8-001, was synthesized and the compound GC-376 was purchased to serve as control compounds for selectivity comparison side-by-side with the prototype inhibitor described herein. The synthesis of the important acyoxymethyl ketone P1 glutamine mimetic, Compound E (AP-7-297B) was accomplished using the reported synthesis starting from N-Boc L-glutamine di-methyl ester as shown (Scheme 2) (Dolle et al., 1997; Dolle et al., 1994; Krantz et al., 1991; Jain & Vederas, 2004). Synthesis of the amides AP-8-011 and AP-8-012 was accomplished starting from intermediate D (Scheme 1) which was deprotected to provide the primary amine and then coupled with the corresponding carboxylic acids. These were hydrolyzed and the resulting acids were reacted with the acyoxymethyl ketone P1 glutamine mimetic, E (Table 1).

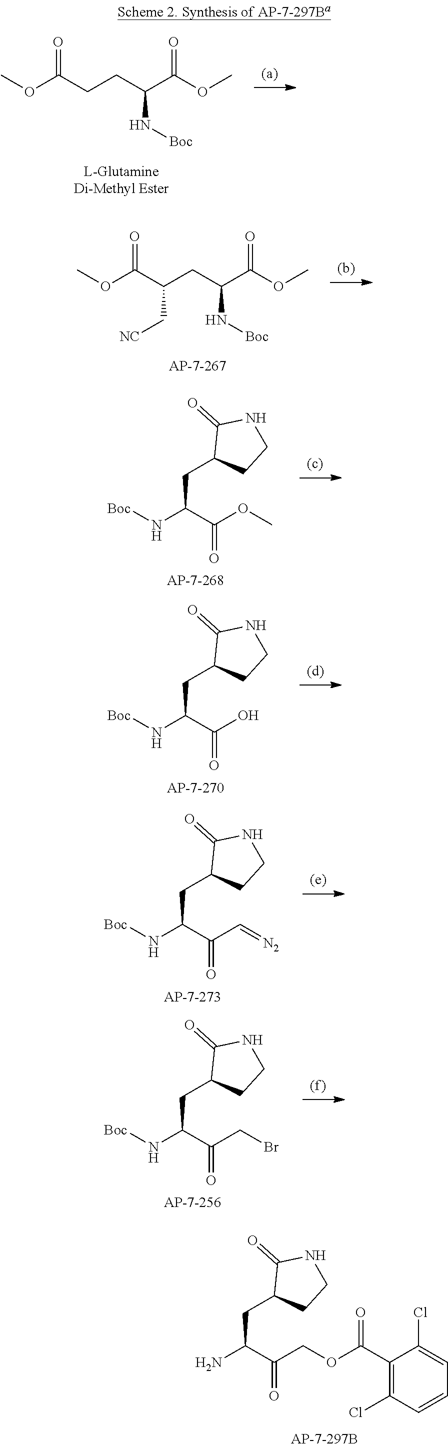

^{a}Reagents and conditions: (a) 2-bromoacetonitrile, Lithium bis(trimethylsilyl)amide solution 1.0 M in THF, THF, −78° C.—rt, 2 h. (b) i. H_{2(g)}, Pt₂O, MeOH, rt, 24 h. ii. MeOH, rt, 24 h. (c) 20% aq NaOH in MeOH:THF:H₂O (5:5:1), it, 4 h. (d) Isobutyl chloroformate, THF, Et₃N, −30° C.-10° C., 1 h then CH₂N₂ in Et₂O, 0° C.—rt, 16 h. (e) 33% HBr in H₂O, THF, −20° C. (f) i. Cesium fluoride, 2,6-dichlorobenzoic acid, THF.

TABLE 1
IC50 Values of AP-8-013, analogs, and controls.

| | Compound | MPro | CatB | CatL | Thrombin |
|---|---|---|---|---|---|
| Structure | ID | IC50, nM | IC50, nM | IC50, nM | IC50, nM |
| 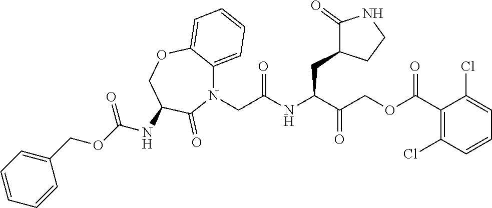 [SMILES: O=C(CN1C(=O)[C@@H](NC(=O)OCc2ccccc2)COc2ccccc21)N[C@@H](C[C@@H]1CCNC1=O)C(=O)COC(=O)c1c(Cl)cccc1Cl] | AP-8-013 | 230 ± 18 | 18,000 ± 9100 | >32000 | >32,000 |
| 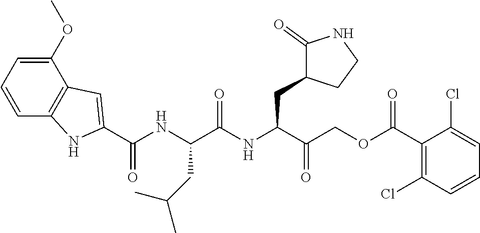 [SMILES: COc1cccc2[nH]c(C(=O)N[C@@H](CC(C)C)C(=O)N[C@@H](C[C@@H]3CCNC3=O)C(=O)COC(=O)c3c(Cl)cccc3Cl)cc12] | AP-8-001 | 11 ± 0.7 | 24 ± 7.5 | 1.8 | >10,000 |
| 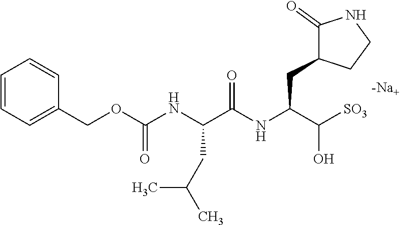 | GC-376 | 18 ± 1.5 | 37 ± 15 | 0.05 | >10,000 |
| 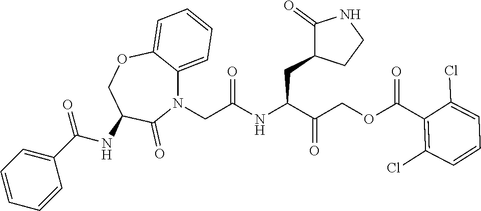 [SMILES: O=C(CN1C(=O)[C@@H](NC(=O)c2ccccc2)COc2ccccc21)N[C@@H](C[C@@H]1CCNC1=O)C(=O)COC(=O)c1c(Cl)cccc1Cl] | AP-8-011 | 620 ± 48 | 13,000 ± 3600 | >32000 | >32,000 |
| 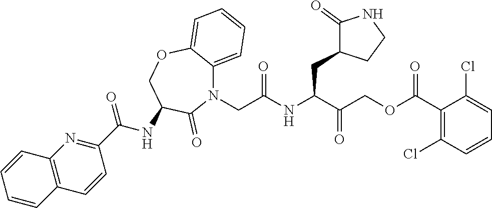 [SMILES: O=C(CN1C(=O)[C@@H](NC(=O)c2ccc3ccccc3n2)COc2ccccc21)N[C@@H](C[C@@H]1CCNC1=O)C(=O)COC(=O)c1c(Cl)cccc1Cl] | AP-8-012 | 580 ± 40 | 1800 ± 260 | 23,058 | >32,000 |

^{a}Enzyme assay. Values were calculated from at least 10 point dose-response curves performed in duplicate.

### Example 2—Biological Evaluation

To initially assess biological activity, a continuous, fluorescence-based Mpro enzymatic assay was developed which monitored the cleavage of the fluorescently quenched substrate DABCYL-Lys-HCoV-SARS replicase polyprotein 1ab (3235-3246)-Glu-EDANS (Ma et al., 2020; Tietjen et al., 2021). Upon cleavage of the substrate, an increase in fluorescence of EDANS was observed at 355/490 nm, which in turn may be inhibited with co-incubation with Mpro inhibitors. Limits of protein and time linearity, substrate Kₘ and Vₘₐₓ, tolerance of 1% DMSO, reproducibility of screening, and titrations of reference compounds were conducted for assay validation (see Example 5). The Kₘ for the substrate was observed to be 30 μM. At 6 μM substrate, the reaction was linear for 30 min with up to 100 nM Mpro. Based on these observations, 5 μM substrate and 50 nM Mpro concentrations were chosen for compound assessment. The effect of 1% DMSO on Mpro activity was tested and found to have no negative effects on the assay. However, compounds with high fluorescence background did interfere in the assay, therefore background fluorescence was tested separately (Z′ score=0.83). GC-376 was tested as a positive control and was shown to have a half-maximal inhibitory concentration (IC₅₀) of 18 nM, consistent with reported values (Table 1) (Ma et al., 2020; Tietjen et al., 2021). Using this assay, the inhibitors were assessed and it was confirmed that AP-8-013 exhibited dose-dependent inhibition of Mpro with an IC₅₀ of 230±18 nM (Table 1). While derivatives AP-8-011 and AP-8-012 had comparable efficacies (IC₅₀s=620±48 and 580±40 nM, respectively), all derivatives exhibited at least 20-fold lower activities compared to control compound AP-8-001 (IC₅₀=11 t 0.7 nM; Table 1). AP-8-013 and derivatives required a 1-2 h incubation time to achieve maximum inhibition (Strelow 2017) which, without being bound by theory, suggested that the highly constrained conformation required longer incubation times for binding to the substrate pocket in contrast to AP-8-001 or GC-376 which achieved maximum inhibition within 10 min. Moreover, while AP-8-001 also inhibited the enzymatic activity of cysteine proteases Cathepsin B and L with comparable or improved activities (IC₅₀s=24±7.5 and 1.8±0.27 nM, respectively), no substantial inhibition of these proteases was observed in the presence AP-8-013 (IC₅₀s>15 μM; Table 1).

No activity was observed against the thrombin serine protease (IC₅₀>32 μM; Table 1), indicating high selectivity of AP-8-013 for Mpro. As these host proteases are essential for processing host peptides (Turk et al., 2012). into their mature form, AP-8-013 and derivatives have a more attractive biological activity profile as they are, without being bound by any theory, expected to have less effect on host protease activities, host protease-mediated cellular homeostasis, and overall safety.

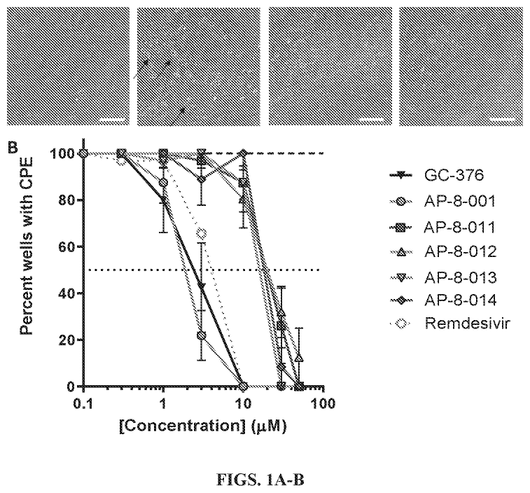

To confirm cellular antiviral activity, a cytopathic effect (CPE)-based assay with infectious SARS-CoV-2 in Vero-E6 cells was used (Tietjen et al, 2021). Briefly, Vero-E6 cells were treated with compounds for 2 hours in 8-fold replicates in 96-well format before infection with 50× median tissue culture infectious dose (TCID50) of SARS-CoV-2 (USA-WA1/2020 variant). Cells were then incubated for 4 days with daily scoring of CPE across all wells by a user blinded to experimental conditions. Using the approach, widespread CPE, identified as cell rounding and cell death, was observed at 4 days' post infection (FIG. 1A). However, in the presence of low micromolar concentrations of GC-376 or AP-8-001, CPE was completed inhibited in these cultures with dose-dependence, with a calculated half-maximal effective concentration (EC₅₀) of 3.4±0.9 and 1.2±0.3 μM, respectively (FIG. 1; Table 2). Comparable activity with nucleoside analog remdesivir (EC₅₀=3.1±0.7 μM; FIG. 1B) was observed (Case et al, 2020). Good inhibition with all test compounds was observed. For example, an EC₅₀ of 12.8±1.6 μM for AP-8-013 (Table 2) was obtained, which is only 3.8-fold reduced potency compared to GC-376 and 10.7-fold vs. AP-8-001 (EC₅₀=1.2±0.3 μM; FIG. 1B). No evidence of cytotoxicity was observed with up to 100 μM of any compound, as measured by resazurin staining following 4 days' treatment in uninfected Vero-E6 cells.

TABLE 2
EC50 values of AP-8-013, analogues, and controls in viral CPE assays.

| EC50 (μM) | GC-376 | AP-8-001 | AP-8-011 | AP-8-012 | AP-8-013 | AP-8-014 | Remdesivir |
|---|---|---|---|---|---|---|---|
| USA-WA1/2020 | 3.4 ± 0.9 | 1.2 ± 0.3 | 14.1 ± 3.6 | 16.3 ± 8.1 | 12.8 ± 1.6 | 11.7 ± 1.1 | 3.1 ± 0.7 |
| B.1.1.7 | 1.3 ± 0.6 | | | | 10.6 ± 1.0 | | |
| B.1.351 | 1.2 ± 0.6 | | | | 5.5 ± 0.4 | | |

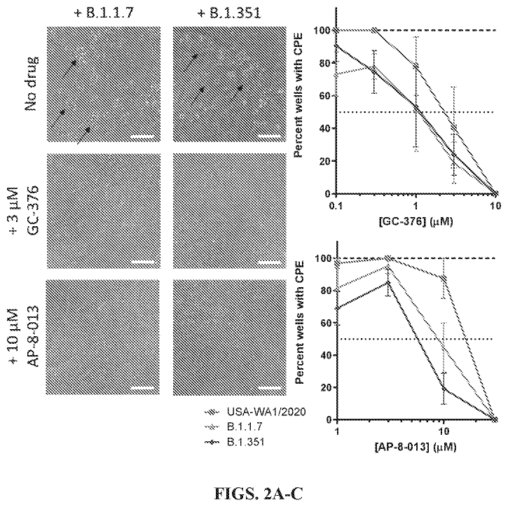

When the ability of AP-8-013 and control GC-376 to inhibit CPE due to SARS-CoV-2 VOC including B.1.1.7/Alpha and B.1.351/Beta variants was testing, it was found that both maintained antiviral activities similar to or improved over those observed in cells infected with the USA-WA1/2020 variant (FIGS. 2A-C). For example, GC-376 inhibited both B.1.1.7 and B.1.351 variants in this assay with EC₅₀s of 1.3±0.6 and 1.2±0.6 μM, respectively, compared to 3.3±1.2 μM against the initial USA-WA1/2020 variant (FIG. 2B; Table 2). Similarly, AP-8-013 exhibited EC₅₀s of 10.6±1.0 and 5.5±0.4 μM against B.1.1.7 and B.1.351 variants, compared to 12.8±1.6 μM vs. USA-WA1/2020 (FIG. 2C; Table 2).

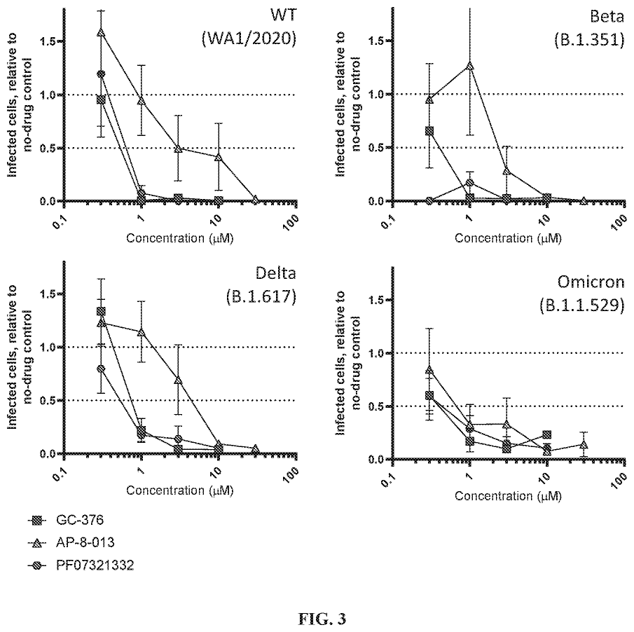

The effects were also studied to determine the inhibition of SARS-CoV-2 replication in VeroE6 cells. Briefly, Vero-E6 cells were plated at 5,000 cells in 384-well format and incubated for 24 hours. Following incubation, cells were treated with compounds at defined concentrations and infected with 150× TCID50 of WT, Beta, Delta, or Omicron variant SARS-CoV-2. Infected cells were incubated for 48 hours, fixed in a final concentration of 4% paraformaldehyde for at least 30 minutes to inactivate virus, and immunostained using primary anti-SARS-CoV-2 nucleocapsid primary antibody (HL344; GeneTex, Irvine, CA) at a 1:1000 concentration and goat-anti-rabbit IgG Alexa Fluor 555 secondary antibody at a 1:2000 concentration (Thermo Fisher, Waltham, MA). Cells were also counterstained with 1 μg/mL Hoechst. High-content imaging was then performed across 9 non-overlapping images per well using a Nikon Eclipse Ti inverted microscope and Nikon NIS Elements AR software v. 5.30.02 (Nikon Amdericas, Inc., Melville, NY). For each image, cell nuclei and Alexa fluor 555-positive cells were counted, with Alexa-Fluor 555-positive cells counted as a percentage of total nuclei within each image. Results denote them mean+/−s.e.m. from 5 independent experiments. In all cases, AP-8-013 inhibits virus replication with EC₅₀s<10 μM, although these activities do not reach those of control Mpro inhibitors. See FIG. 3 and Table 3 below.

TABLE 3
Half-maximal effective concentrations (EC₅₀s) of
Mpro inhibitors against virus replication above.

| WT (WA1/2020) | EC50 (μM) | s.e.m. | Beta (B.1.351) | EC50 (μM) | s.e.m. |
|---|---|---|---|---|---|
| GC-376 | 0.27 | 0.48 | GC-376 | 0.29 | 0.5 |
| AP-8-013 | 2.12 | 2.1 | AP-8-013 | 3.07 | 11 |
| PF07321332 | 0.38 | 0.91 | PF07321332 | n/d | |

| Delta (B.1.617) | EC50 (μM) | s.e.m. | Omicron (B.1.1.529) | EC50 (μM) | s.e.m. |
|---|---|---|---|---|---|
| GC-376 | 0.64 | 2.2 | GC-376 | 0.36 | 0.54 |
| AP-8-013 | 3.04 | 2.4 | AP-8-013 | 0.91 | 0.91 |
| PF07321332 | 0.33 | 0.28 | PF07321332 | 0.44 | 0.16 |

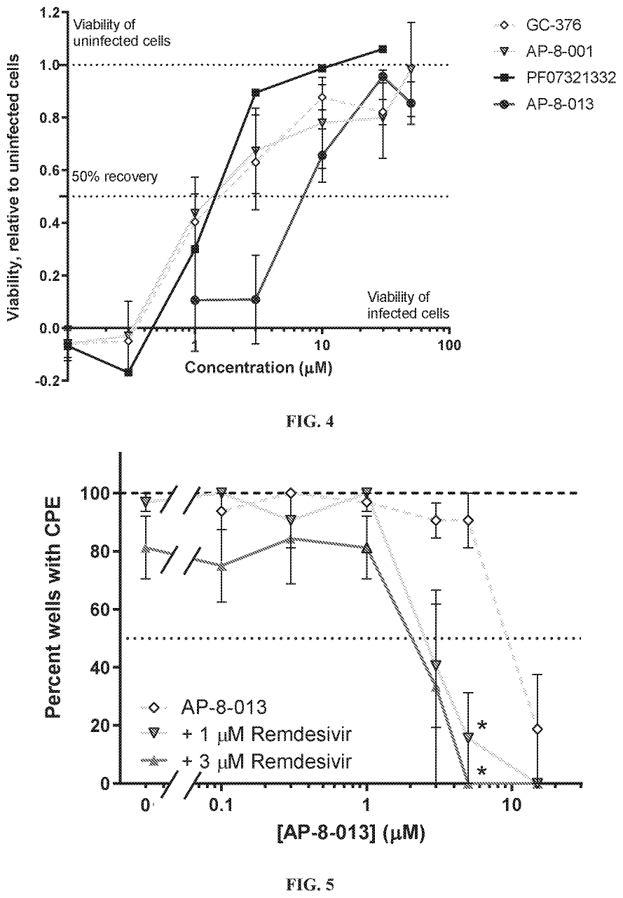

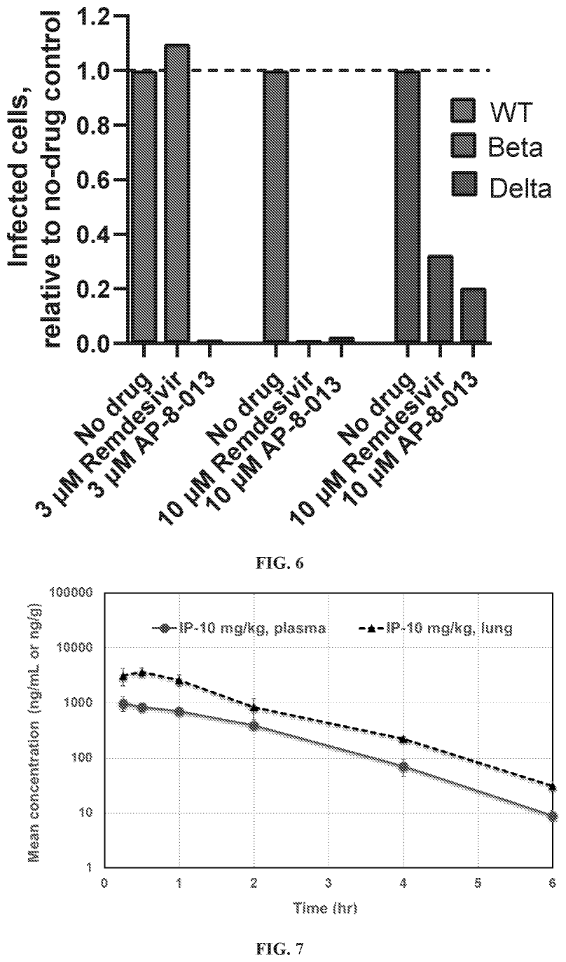

Briefly, Vero-E6 cells were plated at 20,000 cells in 96-well format and incubated for 24 hours. Following incubation, cells were treated with compounds at defined concentrations in infected with 150× of SARS-CoV-2 (WT virus, WA1/2020). Infected cells were incubated for 96 hours before treatment with alamar blue to a final concentration of 20 μg/mL for 4 hours to measure cell viability. Cells were then fixed in 4% paraformaldehyde for at least 30 minutes to inactivate virus, and alamar blue fluorescence intensity was measured using a ClarioStar plate reader (BMC Labtech). Background fluorescence was subtracted from cells containing media and resazurin but no cells. Data were normalized to viability of uninfected cells (1.0) and viability of infected cells with no drug added (0.0). Data show the mean+/−s.e.m. from at least 3 independent experiments. Results show that AP-8-013 inhibits virus replication with an EC₅₀<10 μM, although these activities do not reach those of control Mpro inhibitors. See FIG. 4 and Table 4 below.

TABLE 4
Half-maximal effective concentrations (EC₅₀s) of
Mpro inhibitors against virus replication above

| WT (WA1/2020) | EC50 (μM) | s.e.m. |
|---|---|---|
| GC-376 | 2.05 | 1.7 |
| AP-8-001 | 1.97 | 1.6 |
| AP-8-013 | 7.25 | 6.6 |
| PF07321332 | 1.57 | 2.3 |

### Example 3—Combinations Support Synergistic Effects

Mpro, without being bound by any theory, processes the viral polypeptide pp1ab which encodes for essential non-structural proteins important in viral replication and transcription, including the RNA-dependent RNA polymerase (Thiel et al., 2003), which is the viral target of remdesivir. The CPE assay was used to evaluate AP-8-013 at sub-optimal antiviral doses (0.1 to 5 μM) in combination with sub-optimal anti-viral doses of remdesivir (0.1 to 3 μM) in USA-WA1/2020 variant SARS-CoV-2-infected Vero-E6 cells to evaluate potential synergistic effects when applied in combination (FIG. 5). Notably, enhanced CPE inhibition was observed when either 3 or 5 μM AP-8-001 was combined with 1 or 3 μM remdesivir, none of which were effective inhibiting CPE when added alone (FIG. 5). For example, when assessed across 5 independent experiments of 8-fold replicates, single treatments of either 3 μM AP-8-013 or 3 μM remdesivir inhibited CPE in only an average of 7.5 t 5.0% and 15.0±9.2% of wells, respectively. In contrast, co-incubation with both 3 μM AP-8-013 and 3 μM remdesivir inhibited all CPE across all wells (100 t 0.0%; FIG. 5), resulting in a 4.4-fold increased inhibition relative to what would be expected if AP-8-013 and remdesivir acted by strictly additive effects (i.e., 22.5% inhibition). Similarly, while 1 μM remdesivir inhibited CPE in only 2.5±2.5% wells across 5 independent 8-replicate experiments, additional co-incubation with 3 μM AP-8-013 inhibited CPE in 84.4±22.1% of wells (FIG. 5), corresponding to an 8.4-fold increased activity over expected additive effects (i.e., 10.0% inhibition). These levels of synergism were statistically significant (p=0.02 for both comparisons; Student's paired t-test) as measured by the Bliss independence model of evaluating drug combination activities (Richard et al., 2020). These results indicate that low-doses of AP-8-013 and remdesivir which are ineffective on their own can combine to synergistically inhibit SARS-CoV-2 replication, which may further potentially minimize potential off-target effects of individual compounds when applied as monotherapy. In contrast, no obvious changes in inhibition were observed when remdesivir was added to concentrations of AP-8-013 below 3 μM (FIG. 5). Addition of less than 1 μM remdesivir also did not improve the antiviral activity of AP-8-013 at any concentration.

### Example 4—ADME/PK Evaluation

AP-8-013, AP-8-001, AP-8-011, AP-8-012, and the non-Cbz protected AP-8-013, AP-9-055 were evaluated for metabolic stability (Chempartner, Shanghai, China) by incubation in mouse liver microsomes. Unfortunately, all these analogs show poor stability in this assay with half-life (T_{1/2}) less than 2 minutes. The acyloxymethyl ketone is suspected to be the key metabolic liability. AP-8-013 was evaluated in a male hamster pharmacokinetic study (Chempartner, Shanghai, China) to determine plasma and lung concentration levels over time. The compound was administered as a single dose at 10 mg/kg via intraperitoneal injection formulated in 10% DMSO/10% Solutol® HS15/PBS at 2 mg/mL (FIG. 7). There were no abnormal clinical symptoms observed during the in-life phase. Interestingly, the lung AP-8-013 concentration was 320 percent higher than plasma (AUC_{lung}/AUCₚₗₐₛₘₐ) achieving a concentration of about 8 μM at 10 mg/kg, suggesting that doses of 25 mg/kg or 50 mg/kg should provide compound levels close to those showing efficacy in the CPE assay.

### Example 5—Materials and Experimental Methods

General Procedures. All solvents and chemicals were used as purchased without further purification. The progress of all reactions was monitored on Merck precoated silica gel plates (with fluorescence indicator UV254) using the solvent system indicated. Column chromatography was performed with silica gel 60 (230-400 mesh ASTM) or performed using an automated Biotage Isolera one automated flash purification system with the solvent mixtures specified in the corresponding experiment. TLC plates were visualized by irradiation with ultraviolet light (254 nm). Proton (¹H) and carbon (¹³C) NMR spectra were recorded on a Bruker AVANCE 111400 High Performance Digital NMR Spectrometer using DMSO-_{d6} as solvent. Chemical shifts are given in parts per million (ppm) (δ relative to residual solvent peak for ¹H and ¹³C). Compound purity was determined by LCMS and NMR. LCMS was obtained on a Waters Acquity QDa LCMS mass spectrometer. Purity of all final compounds was 95% or higher

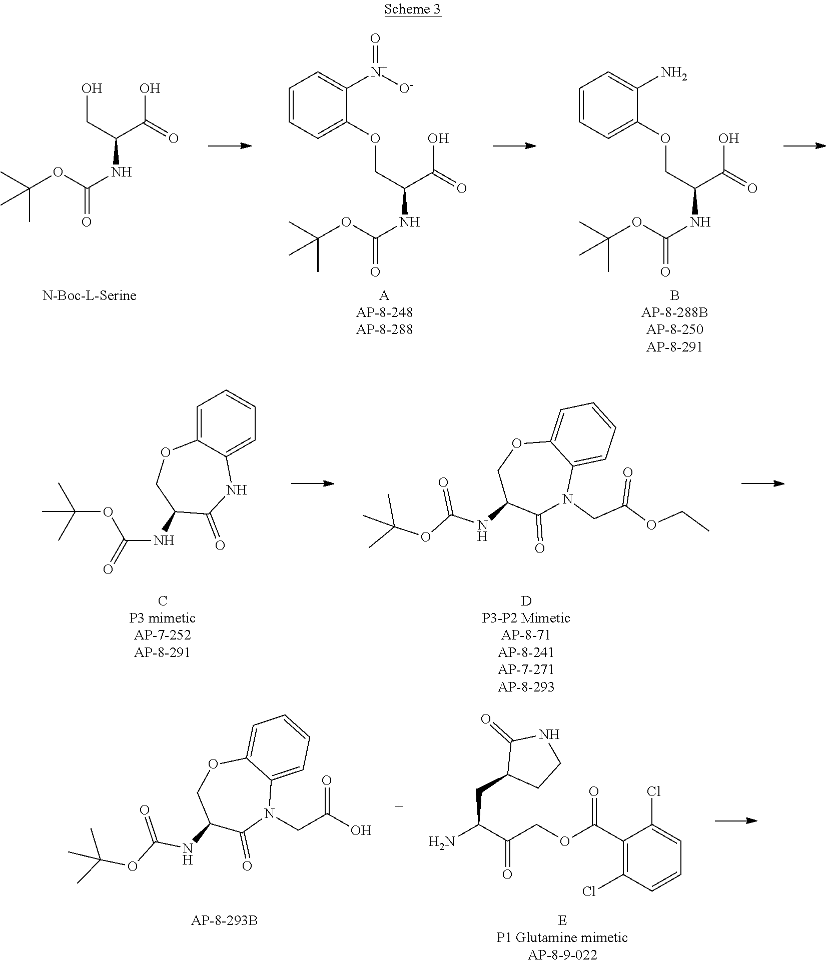

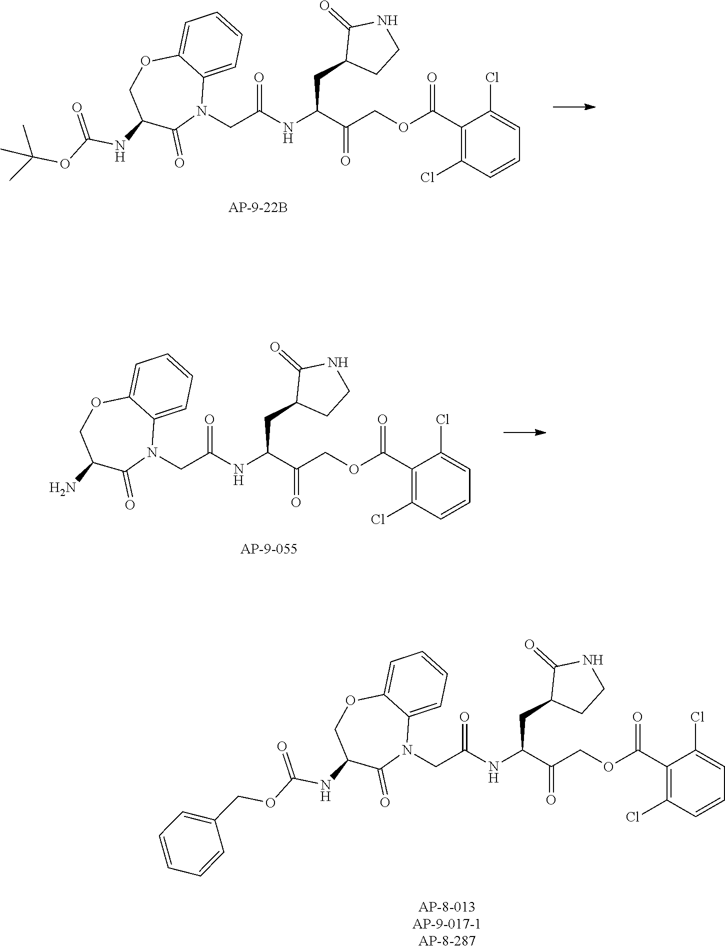 [SMILES: CC(C)(C)OC(=O)N[C@H]1COc2ccccc2N(CC(=O)N[C@@H](C[C@@H]2CCNC2=O)C(=O)COC(=O)c2c(Cl)cccc2Cl)C1=O ; N[C@H]1COc2ccccc2N(CC(=O)N[C@@H](C[C@@H]2CCNC2=O)C(=O)COC(=O)c2c(Cl)cccc2Cl)C1=O ; O=C(CN1C(=O)[C@@H](NC(=O)OCc2ccccc2)COc2ccccc21)N[C@@H](C[C@@H]1CCNC1=O)C(=O)COC(=O)c1c(Cl)cccc1Cl]

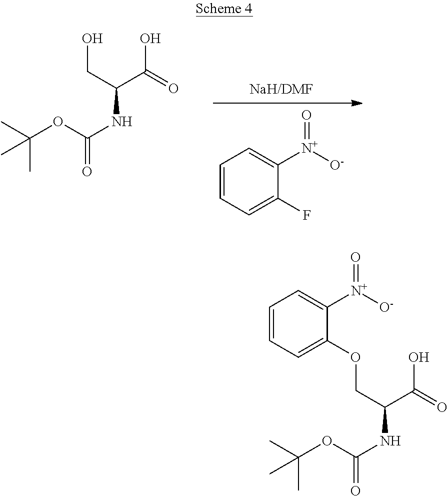 [SMILES: CC(C)(C)OC(=O)N[C@@H](CO)C(=O)O ; O=[N+]([O-])c1ccccc1F ; CC(C)(C)OC(=O)N[C@@H](COc1ccccc1[N+](=O)[O-])C(=O)O]

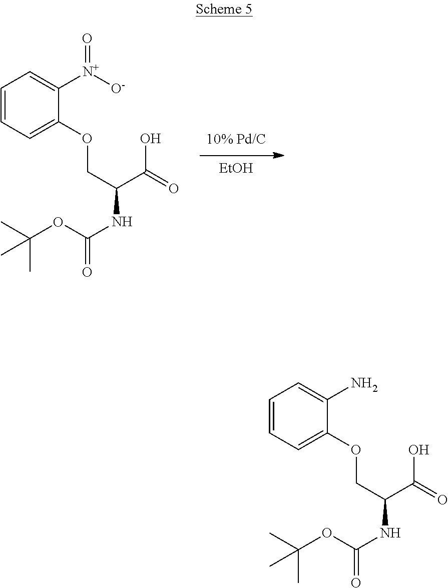 [SMILES: "EtOH" ; CC(C)(C)OC(=O)N[C@@H](COc1ccccc1[N+](=O)[O-])C(=O)O ; CC(C)(C)OC(=O)N[C@@H](COc1ccccc1N)C(=O)O]

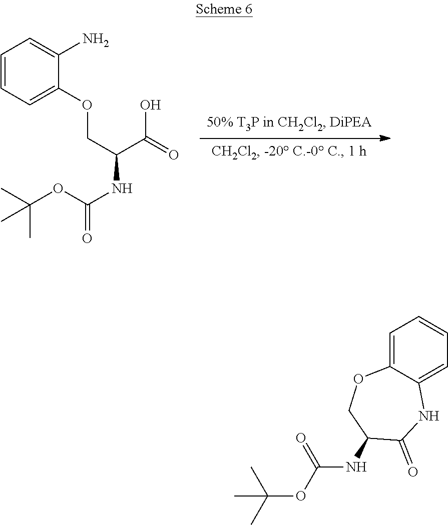 [SMILES: CC(C)(C)OC(=O)N[C@@H](COc1ccccc1N)C(=O)O ; "CH2Cl2, -20° C.-0° C., 1 h" ; CC(C)(C)OC(=O)N[C@H]1COc2ccccc2NC1=O]

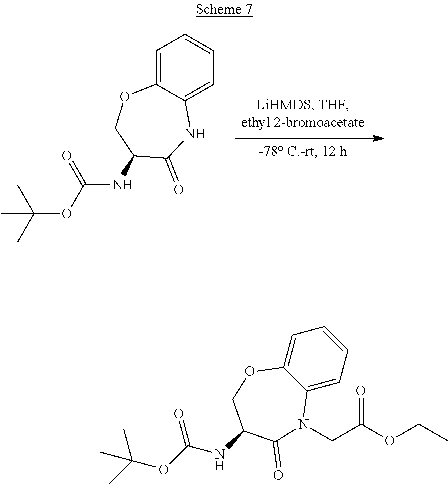 [SMILES: CC(C)(C)OC(=O)N[C@H]1COc2ccccc2NC1=O ; "-78° C.-rt, 12 h" ; CCOC(=O)CN1C(=O)[C@@H](NC(=O)OC(C)(C)C)COc2ccccc21]

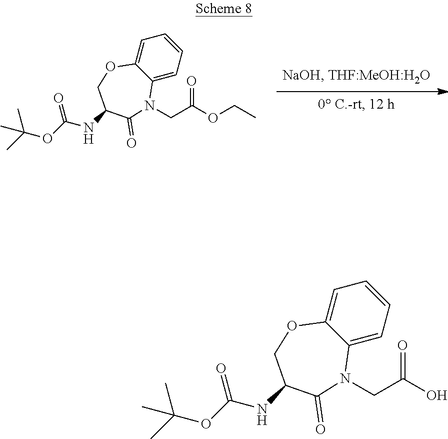 [SMILES: CCOC(=O)CN1C(=O)[C@@H](NC(=O)OC(C)(C)C)COc2ccccc21 ; "0° C.-rt, 12 h" ; CC(C)(C)OC(=O)N[C@H]1COc2ccccc2N(CC(=O)O)C1=O]

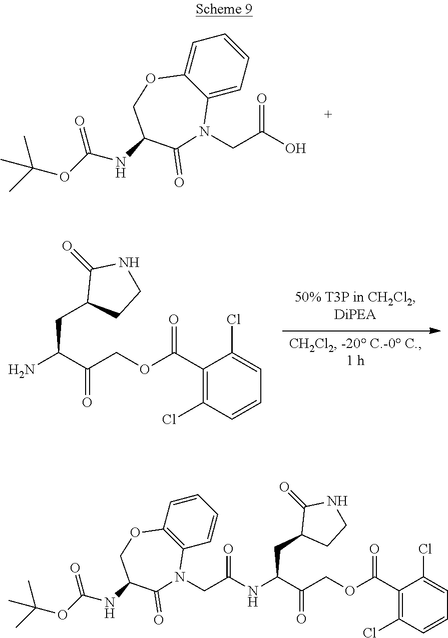 [SMILES: CC(C)(C)OC(=O)N[C@H]1COc2ccccc2N(CC(=O)O)C1=O ; N[C@@H](C[C@@H]1CCNC1=O)C(=O)COC(=O)c1c(Cl)cccc1Cl ; "CH2Cl2, -20° C.-0° C.,1 h" ; CC(C)(C)OC(=O)N[C@H]1COc2ccccc2N(CC(=O)N[C@@H](C[C@@H]2CCNC2=O)C(=O)COC(=O)c2c(Cl)cccc2Cl)C1=O]

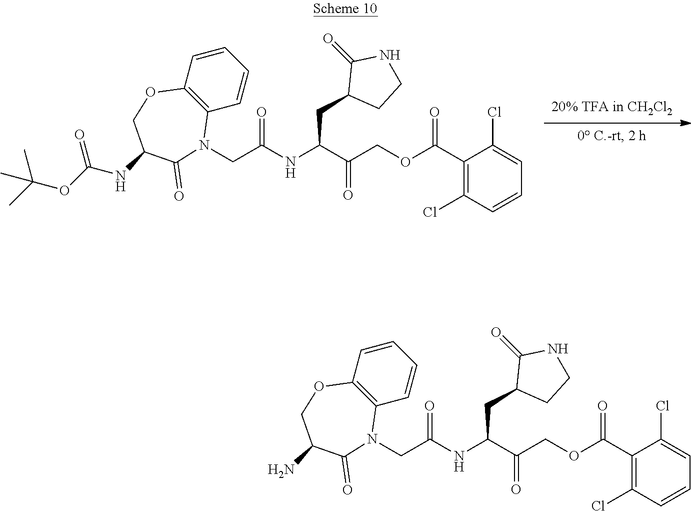 [SMILES: CC(C)(C)OC(=O)N[C@H]1COc2ccccc2N(CC(=O)N[C@@H](C[C@@H]2CCNC2=O)C(=O)COC(=O)c2c(Cl)cccc2Cl)C1=O ; "0° C.-rt, 2 h" ; N[C@H]1COc2ccccc2N(CC(=O)N[C@@H](C[C@@H]2CCNC2=O)C(=O)COC(=O)c2c(Cl)cccc2Cl)C1=O]

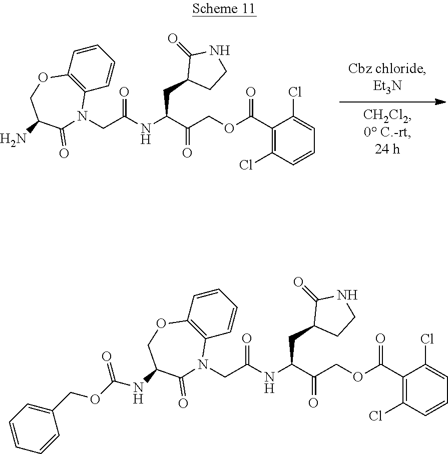 [SMILES: N[C@H]1COc2ccccc2N(CC(=O)N[C@@H](C[C@@H]2CCNC2=O)C(=O)COC(=O)c2c(Cl)cccc2Cl)C1=O ; "CH2Cl2,0° C.-rt,24 h" ; O=C(CN1C(=O)[C@@H](NC(=O)OCc2ccccc2)COc2ccccc21)N[C@@H](C[C@@H]1CCNC1=O)C(=O)COC(=O)c1c(Cl)cccc1Cl]

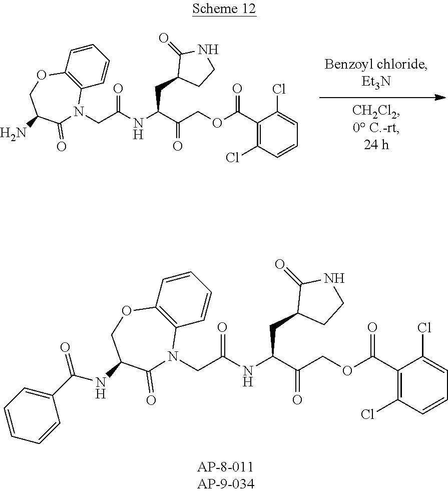 [SMILES: N[C@H]1COc2ccccc2N(CC(=O)N[C@@H](C[C@@H]2CCNC2=O)C(=O)COC(=O)c2c(Cl)cccc2Cl)C1=O ; "CH2Cl2,0° C.-rt,24 h" ; O=C(CN1C(=O)[C@@H](NC(=O)c2ccccc2)COc2ccccc21)N[C@@H](C[C@@H]1CCNC1=O)C(=O)COC(=O)c1c(Cl)cccc1Cl]

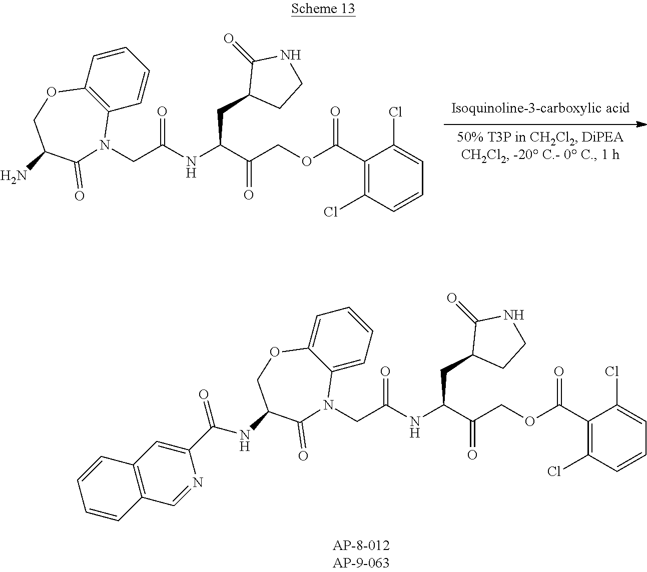 [SMILES: N[C@H]1COc2ccccc2N(CC(=O)N[C@@H](C[C@@H]2CCNC2=O)C(=O)COC(=O)c2c(Cl)cccc2Cl)C1=O ; "50% T3P in CH2Cl2, DiPEACH2Cl2, -20° C.- 0° C., 1 h" ; O=C(CN1C(=O)[C@@H](NC(=O)c2cc3ccccc3cn2)COc2ccccc21)N[C@@H](C[C@@H]1CCNC1=O)C(=O)COC(=O)c1c(Cl)cccc1Cl]

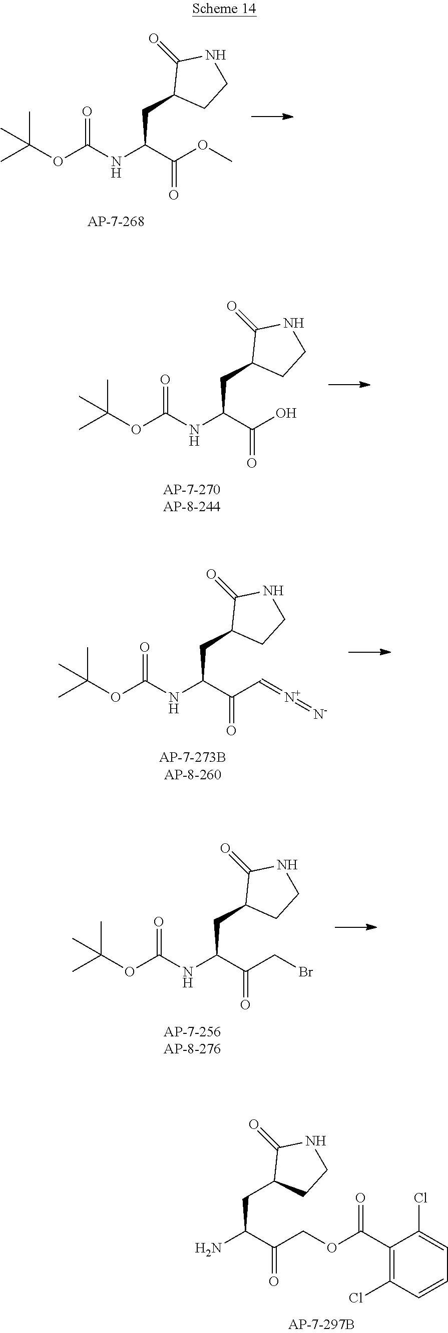 [SMILES: COC(=O)[C@H](C[C@@H]1CCNC1=O)NC(=O)OC(C)(C)C ; CC(C)(C)OC(=O)N[C@@H](C[C@@H]1CCNC1=O)C(=O)O ; CC(C)(C)OC(=O)N[C@@H](C[C@@H]1CCNC1=O)C(=O)C=[N+]=[N-] ; CC(C)(C)OC(=O)N[C@@H](C[C@@H]1CCNC1=O)C(=O)CBr ; N[C@@H](C[C@@H]1CCNC1=O)C(=O)COC(=O)c1c(Cl)cccc1Cl]

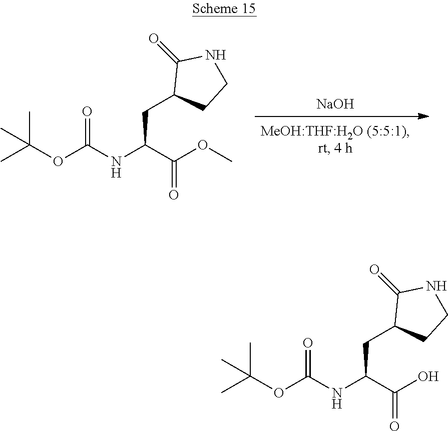 [SMILES: COC(=O)[C@H](C[C@@H]1CCNC1=O)NC(=O)OC(C)(C)C ; "NaOH" ; CC(C)(C)OC(=O)N[C@@H](C[C@@H]1CCNC1=O)C(=O)O]

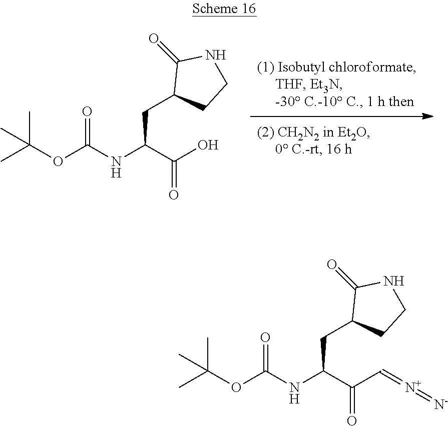

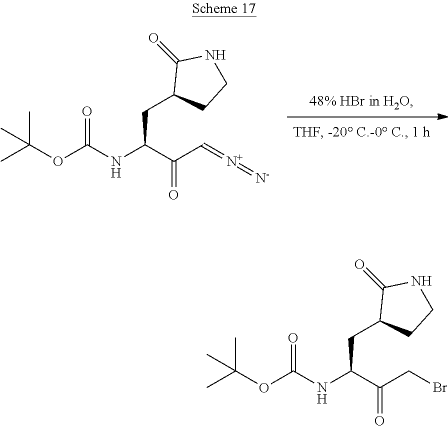 [SMILES: CC(C)(C)OC(=O)N[C@@H](C[C@@H]1CCNC1=O)C(=O)C=[N+]=[N-] ; "48% HBr in H2O," ; CC(C)(C)OC(=O)N[C@@H](C[C@@H]1CCNC1=O)C(=O)CBr]

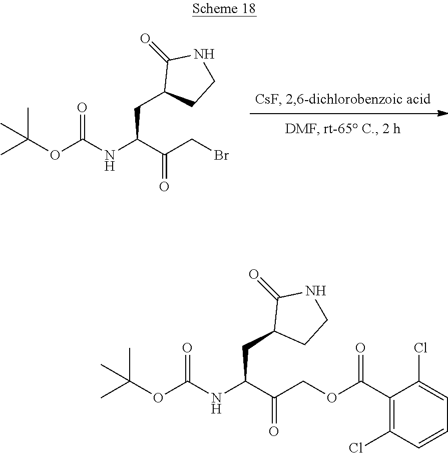 [SMILES: CC(C)(C)OC(=O)N[C@@H](C[C@@H]1CCNC1=O)C(=O)CBr ; "CsF, 2,6-dichlorobenzoic acid" ; CC(C)(C)OC(=O)N[C@@H](C[C@@H]1CCNC1=O)C(=O)COC(=O)c1c(Cl)cccc1Cl]

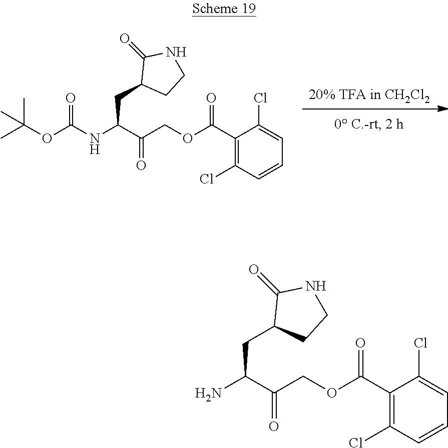 [SMILES: CC(C)(C)OC(=O)N[C@@H](C[C@@H]1CCNC1=O)C(=O)COC(=O)c1c(Cl)cccc1Cl ; "20% TFA in CH2Cl2" ; N[C@@H](C[C@@H]1CCNC1=O)C(=O)COC(=O)c1c(Cl)cccc1Cl]

(S)-2-(Tert-butoxycarbonylamino)-3-(2-nitrophenoxy)propanoic acid: (A); AP-8-248, AP-8-288: To a 0° C. suspension of NaH (60% w/w in mineral oil, 4.74 g, 118.71 mmol) in DMF (50 mL) was added a solution of (S)-2-(tert-butoxycarbonylamino)-3-hydroxypropanoic acid (11.6 g, 56.52 mmol) in DMF (50 mL). After 2 h, a solution of 1-fluoro-2-nitrobenzene (8.77 g, 62.12 mmol) in DMF (25 mL) was added and the resulting mixture was stirred at 0° C. for 4 h. The mixture was poured into 0° C. H₂O (200 mL), acidified to pH 5.0 with 1 N aq HCl, and extracted with EtOAc. The combined organic layers were dried over Na₂SO₄, filtered, and concentrated in vacuo. The crude material was purified by flash chromatography afford title compound (15.76 g, 48.13 mmol, 85%) as thick liquid. The desired product was confirmed by ¹H NMR and MS. ¹H NMR (400 MHz, CDCl₃) δ 7.69 (d, J=35.9 Hz, 1H), 7.49 (d, J=27.4 Hz, 1H), 7.09 (s, 1H), 6.95 (s, 1H), 5.84 (s, 1H), 4.58 (d, J=22.7 Hz, 2H), 4.36 (s, 1H), 1.42 (d, J=27.3 Hz, 9H). ESI-MS (m/z): calculated for C₁₄H₁₉N₂NaO₇ (M+Na)⁺=349.10; found: 349.34. LC-MS m/z: calculated for C₁₄H₁₈N₂NaO₇=349.10; found 349.34.

(S)-3-(2-Aminophenoxy)-2-(tert-butoxycarbonylamino) propanoic acid: (B); AP-8-288B, AP-8-250, AP-8-291: To a stirred solution of (S)-2-(tert-butoxycarbonylamino)-3-(2-nitrophenoxy)propanoic acid (14 g; 42.9 mmol) in 200 mL of EtOH, 10% Pd/C was added (1.36 g 1.29 mmol) under nitrogen atmosphere. Nitrogen gas was switched to hydrogen gas (Balloon pressure) and stirred for 16 h at room temperature. Completion of the reaction was confirmed by LC-MS. Catalyst was removed by passing through a small pad of Celite®. Filtrate was concentrated under reduced pressure to afford the title compound (12.08 g; 40.75 mmol, 95%) as a thick liquid used for the next step without further purification. Product was confirmed by LC-MS.ESI-MS (m/z): calculated for C₁₄H₂₁N₂O₅ (M+H)⁺=297.15; found: 297.27. LC-MS m/z calculated for C₁₄H₂₀N₂O₅=296.14; found [M+1]+=297.27

(S)-Tert-butyl 4-oxo-2,3,4,5-tetrahydrobenzo[b][1,4]oxazepin-3-ylcarbamate: (C); P3 Mimetic; AP-7-252, AP-8-291: To a stirred solution of (S)-3-(2-aminophenoxy)-2-(tert-butoxycarbonylamino)propanoic acid (10.0 g; 33.75 mmol) in 200 mL of dry CH₂Cl₂ at −20° C. was added diisopropyl ethyl amine (24.12 mL; 135.0 mmol) and T3P 50% solution in CH₂Cl₂ by weight (23.6 g; 37.12 mmol) dropwise simultaneously. The reaction mixture was allowed to stir for 1 h at 0° C. Completion of the reaction was confirmed by LC-MS. The reaction mixture was quenched with cold water and the product was extracted with CH₂Cl₂ and dried over anhydrous Na₂SO₄. The solvent was evaporated under reduced pressure and the crude product was purified by flash column chromatography to afford title compound (8.45 g; 30.37 mmol, 90%). The product was confirmed by ¹H NMR and MS. ¹H NMR (400 MHz, DMSO-_{d6}) δ 9.92 (s, 1H), 7.31-6.91 (m, 5H), 4.43-4.03 (m, 3H), 1.33 (s, 9H). ESI-MS (m/z): calculated for C₁₄H₁₈N₂NaO₄ (M+Na)⁺=301.12; found: 301.17. LC-MS m/z calculated for C₁₄H₁₈N₂NaO₄=301.12; found [M+Na]⁺=301.17

(S)-Ethyl 2-(3-(tert-butoxycarbonylamino)-4-oxo-3,4-dihydrobenzo[b][1,4]oxazepin-5(2H)-yl)acetate: AP-8-71, AP-8-241, AP-7-271, AP-8-293: To a stirred solution of (S)-tert-butyl 4-oxo-2,3,4,5-tetrahydrobenzo[b][1,4]oxazepin-3-ylcarbamate (4.0 g; 2.64 mmol) in 100 mL of dry THF at −70° C., 1M in hexane LiHMDS (15.81 mL; 2.9 mmol) was dropwise. Reaction mixture stirred for 30 mins at same temperature ethyl 2-bromoacetate (2.4 g, 2.9 mmol) in 10 ml dry THF was added dropwise. Reaction mixture slowly brought to RT and stirred for 12 h. Completion of the reaction was confirmed by LC-MS. Reaction mixture was quenched with aq ammonium chloride at −20° C. The product was extracted with ethyl acetate, the organic layer washed with brine solution and dried over anhydrous Na₂SO₄. The solvent was evaporated under reduced pressure and the crude product was purified by flash column chromatography to afford title compound (4.2 g; 2.1 mmol, 80%) as a white solid. The product was confirmed by ¹H NMR and MS. ¹H NMR (400 MHz, CDCl₃) δ 7.23-7.09 (m, 4H), 5.48 (d, J=6.8 Hz, 1H), 4.80-4.66 (m, 2H), 4.63 (dd, J=9.5, 7.5 Hz, 1H), 4.34-4.14 (m, 4H), 1.40 (s, 9H), 1.34-1.21 (m, 4H). ESI-MS (m/z): calculated for C₁₈H₂₄N₂NaO₆ (M+Na)⁺=387.15; found: 387.32. LC-MS m/z: calculated for C₁₈H₂₄N₂NaO₆=387.15; found [M+Na]⁺=387.32

(S)-Ethyl 2-(3-(benzyloxycarbonylamino)-4-oxo-3,4-dihydrobenzo[b][1,4]oxazepin-5(2H)-yl)acetate: AP-8-293B: To a solution of (S)-ethyl 2-(3-(benzyloxycarbonylamino)-4-oxo-3,4-dihydrobenzo[b][1,4]oxazepin-5(2H)-yl)acetate (3.0 g; 8.23 mmol) in 60 mL of MeOH, H₂O and THF (4:2:1) at room temperature was added LiOH·H₂O (987 mg; 24.70 mmol). The reaction mixture was stirred for 6 h. Completion of the reaction was confirmed by thin layer chromatography. Volatiles were evaporated by reduced pressure, and the crude product was acidified with 1 N HCl at 0° C. to obtain a white precipitate which was filtered and dried under a high vacuum to afford the title compound (2.9 g; 7.95 mmol, 96%) as white solid. The product was confirmed by ¹H NMR and MS. ¹H NMR (400 MHz, DMSO-_{d6}) δ 13.09-12.03 (m, 1H), 7.39 (dd, J=6.0, 3.6 Hz, 1H), 7.34-7.23 (m, 2H), 7.23-7.15 (m, 2H), 4.45 (ddd, J=32.7, 28.3, 17.5 Hz, 3H), 4.30 (d, J=9.5 Hz, 2H), 1.35 (s, 9H). ESI-MS (m/z): calculated for C₁₆H₁₉N₂O₆ (M−H)⁻=335.12; found: 334.95. LC-MS m/z: calculated for C₁₆H₂₀N₂O₆=336.13; found [M−H]⁻=334.95

(S)-3-(2-((S)-3-(tert-butoxycarbonylamino)-4-oxo-3,4-dihydrobenzo[b][1,4]oxazepin-5(2H)-yl)acetamido)-2-oxo-4-((S)-2-oxopyrrolidin-3-yl)butyl 2,6-dichlorobenzoate: AP-9-22B: To a stirred solution of (S)-2-(3-(tert-butoxycarbonylamino)-4-oxo-3,4-dihydrobenzo [b][1,4]oxazepin-5(2H)-yl)acetic acid (464 mg; 0.24 mmol) and (S)-3-amino-2-oxo-4-((S)-2-oxopyrrolidin-3-yl)butyl 2,6-dichlorobenzoate 2,2,2-trifluoroacetate (607 mg; 0.24 mmol) in 20 mL dry CH₂Cl₂ at 0° C. were added diisopropyl ethyl amine (0.0.92 mL, 5.52 mmol) and T3P 50% solution in CH₂Cl₂ by weight (0.982 mg; 1.65 mmol) dropwise simultaneously. The reaction mixture was stirred for an additional 1 h at 0° C., and completion of the reaction was confirmed by LC-MS. The reaction mixture was quenched with cold water and the product was extracted with CH₂Cl₂ and dried over anhydrous Na₂SO₄. The solvent was removed under reduced pressure to provide the crude reaction product, which was purified by flash column chromatography to afford the title compound as white solid (749 mg, 0.19 mmol, 80%) as a white solid. The product confirmed by ¹H NMR and MS. ¹H NMR (400 MHz, CDCl3) δ 9.21 (dd, J=63.9, 5.9 Hz, 1H), 7.52-7.08 (m, 7H), 5.73 (d, J=26.2 Hz, 1H), 5.50 (dd, J=19.0, 7.3 Hz, 1H), 5.17 (dd, J=43.8, 16.8 Hz, 1H), 5.00-4.84 (m, 1H), 4.80-4.66 (m, 1H), 4.66-4.43 (m, 2H), 4.29 (ddd, J=28.1, 24.6, 13.8 Hz, 2H), 3.42-3.14 (m, 2H), 2.42-2.18 (m, 2H), 2.16-1.92 (m, 2H), 1.86-1.63 (m, 2H), 1.49-1.31 (m, 8H). ESI-MS (m/z): calculated for C₃₁H₃₅C₁₂N₄O₉ (M+H)⁺=677.07; found: 677.18. LC-MS m/z: calculated for C₃₁H₃₄Cl₂N₄O₉=676.17; found [M+H]⁺=677.07

(S)-3-(2-((S)-3-amino-4-oxo-3,4-dihydrobenzo[b][1,4]oxazepin-5(2H)-yl)acetamido)-2-oxo-4-((S)-2-oxopyrrolidin-3-yl)butyl 2,6-dichlorobenzoate 2,2,2-trifluoroacetate: AP-9-055: To a stirred solution of (S)-3-(2-((S)-3-(tert-butoxycarbonylamino)-4-oxo-3,4-dihydrobenzo [b][1,4]oxazepin-5(2H)-yl)acetamido)-2-oxo-4-((S)-2-oxopyrrolidin-3-yl)butyl 2,6-dichloro benzoate (370 mg, 0.54 mmol) in 8 mL of CH₂Cl₂ at 0° C. was added 2 mL of TFA. The reaction mixture was then warmed to room temperature and stirred for 2 h. Completion of the reaction was confirmed by LC-MS. Volatiles were evaporated under reduced pressure and the crude product was co-distilled with dry toluene and dried under high vacuum. to afford title compound (378 mg 0.54, 100%). As thick brown color liquid. The product confirmed by ¹H NMR and MS. ¹H NMR (400 MHz, MeOD) δ 7.52-7.41 (m, 4H), 7.41-7.30 (m, 3H), 7.30-7.23 (m, 1H), 5.29-5.10 (m, 2H), 4.87 (d, J=5.8 Hz, 3H), 4.72-4.60 (m, 2H), 4.48 (ddd, J=18.4, 15.9, 9.2 Hz, 3H), 4.30 (dd, J=43.1, 16.7 Hz, 1H), 3.79-3.70 (m, 1H), 3.66 (q, J=4.6 Hz, 2H), 3.60-3.53 (m, 1H), 2.63-2.42 (m, 1H), 2.41-2.24 (m, 2H), 2.14-2.01 (m, 1H), 1.97 (dd, J=9.2, 4.9 Hz, 1H), 1.89-1.78 (m, 1H). ESI-MS (m/z): calculated for C₂₆H₂₇C₁₂N₄O₇ (M+H)⁺=577.13; found [M+H]⁺=577.21 LC-MS m/z: calculated for C₂₆H₂₆Cl₂N₄O₇=576.12; found [M+H]⁺=577.21

(S)-3-(2-((S)-3-(benzyloxycarbonylamino)-4-oxo-3,4-dihydrobenzo[b][1,4]oxazepin-5(2H)-yl)acetamido)-2-oxo-4-((S)-2-oxopyrrolidin-3-yl)butyl 2,6-dichlorobenzoate: AP-8-013, AP-9-017-1: To a stirred solution of (S)-3-(2-((S)-3-amino-4-oxo-3,4-dihydrobenzo[b][1,4]oxazepin-5(2H)-yl) acetamido)-2-oxo-4-((S)-2-oxopyrrolidin-3-yl)butyl 2,6-dichlorobenzoate 2,2,2-trifluoroacetate (323 mg; 0.47 mmol) in 10 mL of dry CH₂Cl₂ at 0° C. were added triethylamine (0.24 mL; 2.3 mmol) and Cbz chloride (88 mg, 0.51 mmol) dropwise simultaneously. The reaction mixture was allowed to stir for 24 h at 0° C. Completion of the reaction was confirmed by LC-MS. The reaction mixture was quenched with cold water and the product was extracted with CH₂Cl₂ and dried over anhydrous Na₂SO₄. The solvent was evaporated under reduced pressure and the crude product was reverse phase HPLC to afford title compound (150 mg, 0.31 mmol, 65%) as white color solid. The product confirmed by ¹H NMR and MS. ¹H NMR (400 MHz, CDCl₃) δ 9.04-8.53 (m, 1H), 7.53-7.05 (m, 11H), 6.26 (d, J=85.9 Hz, 1H), 5.81 (dd, J=44.7, 7.2 Hz, 1H), 5.23-4.85 (m, 4H), 4.84-4.47 (m, 4H), 4.48-4.10 (m, 2H), 3.21 (dd, J=16.0, 7.7 Hz, 1H), 3.07 (dd, J=15.7, 7.5 Hz, 1H), 2.74-2.53 (m, 1H), 2.41-2.12 (m, 2H), 2.12-1.87 (m, 2H), 1.87-1.58 (m, 1H). ESI-MS (m/z): calculated for C34H₃₃Cl₂N₄O₉ (M+H)⁺=711.16; found: 711.15. LC-MS m/z: calculated for C₃₄H₃₂Cl₂N₄O₉=710.15; found [M+H]⁺=711.15

(S)-3-(2-((S)-3-benzamido-4-oxo-3,4-dihydrobenzo[b][1,4]oxazepin-5(2H)-yl)acetamido)-2-oxo-4-((S)-2-oxopyrrolidin-3-yl)butyl 2,6-dichlorobenzoate: AP-8-011, AP-9-034: To a stirred solution of (S)-3-(2-((S)-3-amino-4-oxo-3,4-dihydrobenzo[b][1,4]oxazepin-5(2H)-yl)acetamido)-2-oxo-4-((S)-2-oxopyrrolidin-3-yl)butyl 2,6-dichlorobenzoate hydrochloride (30 mg; 0.05 mmol) in 5 mL of dry CH₂Cl₂ at 0° C. were added triethylamine (25 mg; 0.25 mmol) and Benzoyl chloride (14 mg, 0.1 mmol) dropwise simultaneously. The reaction mixture was allowed to stir for 24 h at 0° C. Completion of the reaction was confirmed by LC-MS. The reaction mixture was quenched with cold water and the product was extracted with CH₂Cl₂ and dried over anhydrous Na₂SO₄. The solvent was evaporated under reduced pressure and the crude product was reverse phase HPLC to afford title compound (15 mg, 0.02 mmol, 45%) as white color solid. The product confirmed by ¹H NMR and MS. ¹H NMR (400 MHz, CDCl₃) δ 9.56-8.88 (m, 1H), 7.87-7.63 (m, 2H), 7.57-7.46 (m, 1H), 7.46-7.38 (m, 2H), 7.38-7.30 (m, 3H), 7.30-7.23 (m, 3H), 5.46-5.28 (m, 1H), 5.27-5.02 (m, 2H), 5.01-4.53 (m, 4H), 4.35 (dd, J=17.2, 10.1 Hz, 2H), 3.29-3.06 (m, 1H), 2.94 (d, J=6.8 Hz, 1H), 2.65 (d, J=9.1 Hz, 1H), 2.40-2.11 (m, 2H), 2.13-1.90 (m, 2H), 1.87-1.59 (m, 1H). ESI-MS (m/z): calculated for C₃₃H₃₁C₁₂N₄O₈ (M+H)⁺ Calc: 681.15; found: 681.07.

(S)-3-(2-((S)-3-(isoquinoline-3-carboxamido)-4-oxo-3,4-dihydrobenzo[b][1,4]oxazepin-5(2H)-yl)acetamido)-2-oxo-4-((S)-2-oxopyrrolidin-3-yl)butyl 2,6-dichlorobenzoate; AP-8-012: To a stirred solution of (S)-2-(3-(tert-butoxycarbonylamino)-4-oxo-3,4-dihydrobenzo [b][1,4]oxazepin-5(2H)-yl)acetic acid (20 mg; 0.03 mmol) and Isoquinoline-3-carboxylic acid (6 mg, 0.03 mmol) in 20 mL dry CH₂Cl₂ at 0° C. was added diisopropyl ethyl amine (21 mg, 0.04 mmol) and T3P 50% solution in CH₂Cl₂ by weight (17 mg; 1.65 mmol) dropwise simultaneously. The reaction mixture was stirred for an additional 1 h at 0° C., and completion of the reaction was confirmed by LC-MS. The reaction mixture was quenched with cold water and the product was extracted with CH₂Cl₂ and dried over anhydrous Na₂SO₄. The solvent was removed under reduced pressure to provide the crude reaction product, which was purified by flash column chromatography to afford the title compound as white solid (10 mg, 42%) as a white solid. The product confirmed by ¹H NMR and MS. ¹H NMR (400 MHz, CDCl₃) δ 9.25 (d, J=7.8 Hz, 1H), 9.10 (d, J=7.5 Hz, 1H), 8.74 (d, J=6.1 Hz, 1H), 8.53 (d, J=4.6 Hz, 1H), 8.09 (d, J=7.9 Hz, 1H), 7.99 (t, J=7.3 Hz, 1H), 7.88-7.68 (m, 2H), 7.41 (d, J=5.0 Hz, 1H), 7.37-7.16 (m, 7H), 6.88 (d, J=34.7 Hz, 1H), 5.37 (dd, J=7.5, 3.6 Hz, 1H), 5.15 (q, J=16.8 Hz, 1H), 4.90-4.64 (m, 3H), 4.48 (ddd, J=30.9, 17.7, 11.7 Hz, 2H), 3.19 (s, 2H), 2.99 (d, J=7.3 Hz, 1H), 2.78-2.61 (m, 1H), 2.41-2.25 (m, 1H), 2.26-2.02 (m, 2H), 1.99 (s, 1H), 1.91-1.75 (m, 1H), 1.76-1.58 (m, 1H). ESI-MS (m/z): calculated for C₃₆H₃₂C₁₂N₅O₈ (M+H)⁺ Calc: 732.16; found: 732.14.

Starting Material: AP-7-268: ¹H NMR (400 MHz, CDCl₃) δ 6.78 (s, 1H), 5.63 (d, J=8.2 Hz, 1H), 4.37-4.23 (m, 1H), 3.74 (s, 3H), 3.43-3.24 (m, 2H), 2.59-2.36 (m, 2H), 2.19-2.06 (m, 1H), 1.94-1.72 (m, 2H), 1.44 (s, 9H). ESI-MS (m/z): C₁₃H₂₂N₂NaO₅ (M+Na)⁺ Calc: 309.14; found: 309.12.

(S)-2-(tert-butoxycarbonylamino)-3-((S)-2-oxopyrrolidin-3-yl)propanoic acid: AP-7-270, AP-8-244: To a solution (S)-methyl 2-(tert-butoxycarbonylamino)-3-((S)-2-oxopyrrolidin-3-yl) propanoate (AP-7-268) purchased from Aaron chemicals (10 g; 34.92 mmol) in 180 mL of MeOH, H₂O and THF (5:5:1) at room temperature was added LiOH·H₂O (4.39 mg; 104.7 mmol). The reaction mixture was stirred for 6 h. Completion of the reaction was confirmed by thin layer chromatography. Volatiles were evaporated by reduced pressure, and the crude product was acidified with 1 N HCl at 0° C. and saturated with NaCl product extracted with 20% IPA in CH₂Cl₂ (100 ml×4) dried over Na₂SO₄, filtered, and concentrated to afford the title compound (9.1 g; 33.41 mmol, 95%) as white solid. The product confirmed by ¹H NMR and MS. ¹H NMR (400 MHz, MeOD) δ 7.30-6.94 (m, 2H), 4.14 (dd, J=11.4, 3.7 Hz, 1H), 3.41-3.24 (m, 2H), 2.63-2.42 (m, 1H), 2.42-2.32 (m, 1H), 2.18-2.02 (m, 1H), 1.91-1.68 (m, 2H), 1.44 (s, 9H). ESI-MS (m/z): calculated for C₁₂H₁₉N₂O₅ (M−H)⁺ Calc: 271.14; found: 271.15. LC-MS m/z: calculated for C₁₂H₂₀N₂O₅=272.30; found [M−H]⁻=271.15

tert-butyl (S)-4-diazo-3-oxo-1-((S)-2-oxopyrrolidin-3-yl)butan-2-ylcarbamate: AP-7-273B, AP-8-260: To a stirred solution of N-(tert-butoxycarbonyl)-3-[(3S)-2-oxopyrrolidin-3-yl]-L-alanine (AP-8-260) (7.833 g, 28.76 mmol) in THF (200 mL) was placed under an atmosphere of N₂ and cooled to −20° C. The resulting clear colorless solution was successively treated with triethylamine (6.1 mL, 34.51 mmol) followed by isobutylchloroformate (4.5 mL, 12.0 mmol). The reaction mixture gradually became opaque with a fine white precipitate and after 1 h was filtered. The colorless filtrate was transferred to a nonground joint flask, cooled to 0° C., and slowly treated with a solution of diazomethane (˜35 mL, ˜16.6 mmol) in diethyl ether. Note: The diazomethane was generated employing a Diazald kit according to the procedure described in the Aldrich Technical Bulletin AL-180. The resulting yellow clear solution was gradually warmed to room temperature (rt) over 16 h. At this time, N2 was bubbled through the reaction to remove excess diazomethane followed by in vacuo concentration. The resulting residue was diluted with ethyl acetate (200 mL), washed once with sat. NaHCO₃ (100 mL), once with brine (100 mL), dried over Na₂SO₄, filtered, and concentrated under reduced pressure and the crude product was purified by flash column chromatography to afford title compound (7.67 g; 25.9 mmol, 90%). The product confirmed by ¹H NMR and MS. ¹H NMR (400 MHz, CDCl3) δ 6.10 (s, 1H), 5.79 (s, 1H), 5.62 (s, 1H), 4.24 (s, 1H), 3.35 (dd, J=8.4, 6.3 Hz, 2H), 2.44 (d, J=5.0 Hz, 2H), 2.04 (d, J=6.8 Hz, 1H), 1.95-1.71 (m, 2H), 1.45 (s, 9H). ESI-MS (m/z): calculated for C₁₃H₂₀N₄NaO₄ (M+Na)⁺=319.31; found: 319.26. LC-MS m/z: calculated for C₁₃H₂₀N₄NaO₄=319.14; found [M+Na]⁺=319.26

Tert-butyl (S)-4-bromo-3-oxo-1-((S)-2-oxopyrrolidin-3-yl)butan-2-ylcarbamate: AP-7-276: To a stirred solution of tert-butyl ((1S)-3-chloro-2-oxo-1-([(3S)-2-oxopyrrolidin-3-yl]methyl)propyl)carbamate (3.5 g, 11.81 mmol) in THF (100 mL) at −20° C. under nitrogen was treated with 48% hydrobromic acid (2.2 ml mL, 13.0 mmol) with effervescence observed. The reaction was stirred at 0° C. for 1 h, washed once with water (50 mL), dried over Na₂SO₄, filtered, and concentrated to afford the title compound (3.69 g, 10.51 mmol, 89%) of the title compound as a white solid. Product confirmed by MS.

(S)-3-(tert-butoxycarbonylamino)-2-oxo-4-((S)-2-oxopyrrolidin-3-yl) butyl 2,6-dichlorobenzoate: AP-8-283, AP-9-21: To a stirred solution of tert-butyl (S)-4-bromo-3-oxo-1-((S)-2-oxopyrrolidin-3-yl)butan-2-ylcarbamate (3.69 g, 8.40 mmol) and 2,6-dichlorobenzoic acid (2.5 g, 13.1 mmol) in 60 ml anhydrous DMF at rt under nitrogen atmosphere finely grained anhydrous Cesium fluoride (3.72 g, 25.2 mmol) was reaction temperature raised to 65° C. and stirred for 2 h under nitrogen atmosphere. Completion of the reaction confirmed by LC-MS, the reaction was cooled to RT, diluted with water (100 mL), and with ethyl acetate (200 mL), washed once with brine (100 mL), dried over Na₂SO₄, filtered, and concentrated under reduced pressure and the crude product was purified by flash column chromatography to afford title compound (4.0 g; 6.89 mmol, 82%). The product confirmed by ¹H NMR and MS. ¹H NMR (400 MHz, CDCl₃) δ 7.39-7.20 (m, 3H), 6.84 (s, 1H), 6.44 (d, J=6.8 Hz, 1H), 5.34-4.95 (m, 2H), 4.45 (t, J=10.2 Hz, 1H), 3.38-3.16 (m, 2H), 2.59-2.44 (m, 1H), 2.44-2.28 (m, 1H), 2.18-2.06 (m, 1H), 2.00-1.89 (m, 1H), 1.89-1.66 (m, 1H), 1.46 (s, 9H). ESI-MS (m/z): C₁₈H₁₆Cl₂N₂NaO₄ (M-C₅H₉O₂)⁺ Calc: 380.95; found: 381.04. LC-MS m/z: calculated for C₂₀H₂₄N₂NaO₆=481.09; found [M+Na]⁺=480.95

(S)-3-amino-2-oxo-4-((S)-2-oxopyrrolidin-3-yl) butyl 2,6-dichlorobenzoate 2,2,2-trifluoroacetate: AP-9-022HCl: To a stirred solution of (S)-ethyl 2-(3-(tert-butoxycarbonylamino)-4-oxo-3,4-dihydrobenzo[b][1,4]oxazepin-5(2H)-yl)acetate (500 mg, 1.09 mmol) in 8 mL of CH₂Cl₂ at 0° C. was added 2 mL of TFA. The reaction mixture was then warmed to room temperature and stirred for 2 h. Completion of the reaction was confirmed by LC-MS. Volatiles were evaporated under reduced pressure and the crude product co-distilled with dry toluene dried under high vacuum afforded the title compound (515 g; 109 mmol, 100%). as light brown color thick liquid product used for next step without further purification. The product was confirmed by LC-MS.

Cells, viruses, and reagents. Vero-E6 cells were obtained from the American Tissue Culture Collection and cultured in Dulbecco's Modified Eagle Medium with 4.5 g/L glucose and L-glutamine (Gibco, Gaithersburg, MD), 10% fetal bovine serum (Gemini Bio Products, West Sacramento, CA, USA), 100 U of penicillin/mL, and 100 μg of streptomycin/mL (Sigma Aldrich, St. Louis, MO) at 37° C. and 5% CO₂. The following reagent was deposited by the Centers for Disease Control and Prevention and obtained through BEI Resources, NIAID, NIH: SARS-Related Coronavirus 2, Isolate USA-WA1/2020, NR-52281. The following reagents were obtained through BEI Resources, NIAID, NIH: SARS-Related Coronavirus 2, isolate hCoV-19/England/204820464/2020, NR-54000, contributed by Bassam Hallis, and SARS-Related Coronavirus 2, Isolate hCoV-19/South Africa/KRISP-K005325/2020, NR-54009, contributed by Alex Sigal and Tulio de Oliveria. Remdesivir was purchased from Sigma-Aldrich. GC-376 was purchased from Selleckchem (Houston, TX, USA).

Mpro enzymatic assays. Recombinant Mpro was obtained and Mpro enzymatic assays were performed as previously described (Tietjen et al., 2021). Briefly, 5 μL of 25 nM recombinant Mpro protein was diluted in 25 mM HEPES (pH 7.4), 150 mM NaCl, 5 mM DTT, and 0.005% Tween was dispensed into black 384-well plates. Test compounds were serially diluted into 100% DMSO, and 100 nL was added to Mpro dilutions using a Janus MDT Nanohead (PerkinElmer). Wells were then treated with 5 μL of 5 μM fluorogenic substrate ({DABCYL}-Lys-Thr-Ser-Ala-Val-Leu-Gln-Ser-Gly-Phe-Arg-Lys-Met-Glu-(EDANS)-NH2; Bachem, Vista, CA, USA) and monitored for fluorescence at 355 nm excitation and 460 nm emission every 5 minutes for up to 120 minutes using an Envision plate reader (PerkinElmer). Rate of substrate cleavage was determined using linear regression of the raw data values obtained during the time course. Slopes of these progress curves were then normalized to percent inhibition, where 100% equaled the rate in the absence of Mpro and 0% equaled rate of cleavage in the presence of Mpro and 0.1% DMSO.

Cathepsin L enzymatic assay Assays contained 25 pM cathepsin L (RD systems: 952-CY-010), 5 uM LR-AMC, 100 nL of test compound in 100% DMSO, in at total of 10 uL of 20 mM KPO4, pH 6.0, 150 mM NaCl, 0.005% Tween20, 5 mM DTT in black low volume 384-well plates. The production of AMC was followed at 5 min intervals at 355 nm excitation, 460 nm emission in an Envision microplate reader (PerkinElmer). Reaction rates were determined by linear regression of the resulting progress curves. Rates were normalized to % inhibition, where 0% is equal to the rate in the presence enzyme, and 100% is equal to the rate in the absence of enzyme. Nonlinear regression fits of the data to a one-site dose response curve were performed using XLFit (IDBS).

Cathepsin B enzymatic assay Assays contained 0.6 nM cathepsin B (RD systems: 953-CY-010), 25 uM Z-LR-AMC, 100 nL of test compound in 100% DMSO, in at total of 10 uL of 50 mM MES, pH 5.0, 150 mM NaCl, 0.05% CHAPS, 5 mM DTT in black low volume 384-well plates. The production of AMC was followed at 5 min intervals at 355 nm excitation, 460 nm emission in an Envision microplate reader (PerkinElmer). Reaction rates were determined by linear regression of the resulting progress curves. Rates were normalized to % inhibition, where 0% is equal to the rate in the presence enzyme, and 100% is equal to the rate in the absence of enzyme. Nonlinear regression fits of the data to a one-site dose response curve were performed using XLFit (IDBS).

Thrombin enzymatic assay Assays contained 25 pM thrombin (RD systems: 1473-SE-010), 25 uM BOC—PVR-AMC, 100 nL of test compound in 100% DMSO, in at total of 10 μL of 50 mM Tris, pH 7.0, 100 mM NaCl, 10 mM CaCl2, 0.005% Tween20 in black low volume 384-well plates. The production of AMC was followed at 5 min intervals at 355 nm excitation, 460 nm emission in an Envision microplate reader (PerkinElmer). Reaction rates were determined by linear regression of the resulting progress curves. Rates were normalized to % inhibition, where 0% is equal to the rate in the presence enzyme, and 100% is equal to the rate in the absence of enzyme. Nonlinear regression fits of the data to a one-site dose response curve were performed using XLFit (IDBS).

Resazurin cell viability assay. 2×10⁴ Vero-E6 cells were plated in 96-well plates and incubated before addition of compounds in duplicate, followed by further incubation for an additional 96 hours. Resazurin (Sigma Aldrich) was then added to a final concentration of 20 μg/mL, and cells were incubated for an additional 4 hours. Resazurin-induced fluorescence was then measured using a ClarioStar plate reader (BMG Labtech). Background fluorescence was subtracted from wells containing resazurin and media but no cells and normalized to cells treated with 0.1% DMSO.

Virus generation. 3×10⁶ Vero-E6 cells were incubated in 15 mL of media for 24 hours, replaced with 10 mL fresh media, and incubated with virus at a multiplicity of infection of 0.001. Cells were incubated for 5-7 days until clear CPE was observed throughout the flask. Media was harvested and stored at −80° C. To determine virus titers, Vero-E6 cells were plated in 96-well format at 20,000 cells per well, incubated for 24 hours, and then washed and incubated in fresh media containing 5-fold serial dilutions of thawed virus aliquot, followed by an additional 4 days' incubation. Wells were then scored visually for presence of CPE. TCID50s were then calculated using the Reed-Muench method.

Virus CPE assays. Assays were preformed as described previously (Tietjen et al., 2021). Briefly, Vero-E6 cells were cultured at 20,000 cells/well in 96-well format for 24 hours. Compounds were then added to final concentrations in 8-fold replicates, incubated for a further 2 hours, and then infected with 50× TCID50 of virus. In-plate controls included uninfected cells and infected cells plus 0.1% DMSO in 4-fold replicates. Cells were incubated for an additional 4 days and scored for presence or absence of CPE by a user blinded to the identity of wells.

Data analysis. 50% effective concentrations were calculated using nonlinear regression of a one-side binding model using GraphPad Prism v. 9.1.2 (GraphPad, San Diego, CA, USA). All data are presented as the mean±s.e.m. from at least 3 independent experiments. Synergism from drug combinations was determined using the Bliss independence model as described previously (Richard et al., 2020). Statistical significance for synergy was determined using Student's paired t test, where a two-sided p value of 0.05 was considered significant.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference:
Case et al., Virology, 548:39-48, 2020.
Daniloski et al., Elife, 10:e65365, 2021.
Dolle et al., J Med Chem., 37(23):3863-66, 1994.
Dolle et al., J Med Chem., 39(13):2438-40, 1996.
Dolle et al., J Med Chem., 40(13):1941-6, 1997.
Fan et al., J Biol Chem., 279(3) 1637-42, 2004.
Garcia-Beltran et al., Cell, 184(9):2372-83, 2021.
Hoffman et al., J Med Chem., 63(21):12725-47, 2020.
Itoh et al., I. Chem Pharm Bull (Tokyo), 34(3):1128-47, 1986a.
Itoh et al., III. Chem Pharm Bull (Tokyo)., 34(9):3747-61, 1986b.
Jain & Vederas, Bioorg Med Chem Lett., 14(14):3655-58, 2004.
Krantz et al., Biochemistry, 30(19):4678-87, 1991.
LaPlante et al., J Am Chem Soc., 132(43):15204-12, 2010.
Lee et al., Nat Commun., 11(1):5877, 2020.
Li et al., Cell, 184(9):2362-71, 2021.
Lopez Bernal et al., N Engl J Med., 385(7):585-94, 2021.
Ma et al., Cell Res., 30(8):678-92, 2020.
Planas et al., Nature, 596(7871):276-80, 2021.
Richard et al., J Biol. Chem., 295(42):14804-99, 2020.
Strelow J M. SLAS Discov. 22(1):3-20, 2017.
Thiel et al., J Gen Virol., 84(pt.9):2305-15, 2003
Tietjen et al., Antimicrob Agents Chemother., 65(12):e0077221, 2021.
Turk et al., Biochim Biophys Acta, 1824(1):68-88, 2012.
Ullrich & Nitsche, Boorg Med Chem Lett., 30(17):127377, 2020.
Wang et al., Am J Cancer Res., 10(8):2535-45, 2020.
Wang et al., Nature, 593(7857):130-35, 2021.
Zhang et al., Science, 368(6489):409-412, 2020.
Zhou et al., Cell, 184(9):2348-61, 2021.
Zimmer et al., https://www.nytimes.com/interactive/2020/science/coronavirus-vaccine-tracker.html, 2020.

What is claimed:

1. A compound of the formula: 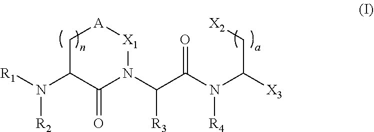
wherein:
A is O, S, or NR′, wherein R′ is hydrogen, alkyl_{(C≤8)}, or substituted alkyl_{(C≤8)};
X₁ is cycloalkanediyl_{(C≤12)}, arenediyl_{(C≤12)}, heteroarenediyl_{(C≤12)}, heterocycloalkanediyl_{(C≤12)}, or a substituted version of any of these groups;
X₂ is heteroarenediyl_{(C≤12)}-R₆, heterocycloalkanediyl_{(C≤12)}-R₆, or a substituted version thereof; or a group of the formula: 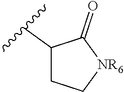
a is 0, 1, or 2;
X₃ is C(O)(CH₂)ₘR₅ or cyano;
m and n are each independently 0, 1, 2, or 3;
R₁ and R₂ are each independently selected from hydrogen, alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; or a monovalent amino protecting group; or R₁ and R₂ are a divalent amino protecting group; or Y₁—Rₐ; wherein:
Y₁ is —C(O)—, —C(O)O—, —C(O)NR_{b}—, —S(O)ₓ—, —S(O)ₓO—, or —S(O)ₓNR_{b′}—; wherein:
x is 0, 1, or 2;
R_{b} and R_{b′} are each independently hydrogen, alkyl_{(C≤12)}, substituted alkyl_{(C≤12)}, acyl_{(C≤12)}, substituted acyl_{(C≤12)}, or a monovalent amino protecting group; and
Rₐ is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
R₃ is hydrogen, alkyl_{(C≤12)}, aryl_{(C≤12)}, aralkyl_{(C≤12)}, heteroaryl_{(C≤12)}, heterocycloalkyl_{(C≤12)}, or a substituted version of any of these groups; or is the side chain of one of the 20 canonical amino acids;
R₄ and R₆ are each independently selected from hydrogen, alkyl_{(C≤12)}, substituted alkyl_{(C≤12)}, acyl_{(C≤12)}, substituted acyl_{(C≤12)}, or a monovalent amino protecting group; and
R₅ is Y₂—R_{c}; wherein:
Y₂ is —NR_{d}—, —S—, —O—, —C(O)—, —OC(O)—, —C(O)O—, —NR_{d}C(O)—, —C(O)NR_{d}—, —OC(O)O—, —OC(O)NR_{d}—, —NR_{d}C(O)O—, —NR_{d}C(O)NR_{d′}—, —S(O)_{y}—, —OS(O)_{y}—, —S(O)—O—, —NR_{d}S(O)_{y}—, —S(O)ₓNR_{d}—, —OS(O)_{y}O—, —OS(O)_{y}NR_{d}—, —NR_{d}S(O)_{y}O—, or —NR_{d}S(O)_{y}NR_{d′}—; wherein:
y is 0, 1, or 2;
R_{d} and R_{d′} are each independently hydrogen, alkyl_{(C≤12)}, substituted alkyl_{(C≤12)}, acyl_{(C≤12)}, substituted acyl_{(C≤12)}, or a monovalent amino protecting group; and
R_{c} is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
or a pharmaceutically acceptable salt thereof.

2. The compound of claim 1 further defined as: 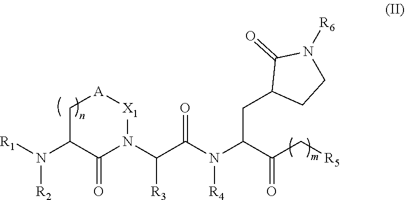
wherein:
A is O, S, or NR′, wherein R′ is hydrogen. alkyl_{(C≤8)}, or substituted alkyl_{(C≤8)};
X₁ is cycloalkanediyl_{(C≤12)}, arenediyl_{(C≤12)}, heteroarenediyl_{(C≤12)}, heterocycloalkanediyl_{(C≤12)}, or a substituted version of any of these groups;
m and n are each independently 0, 1, 2, or 3;
R₁ and R₂ are each independently selected from hydrogen, alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; or a monovalent amino protecting group; or R₁ and R₂ are a divalent amino protecting group; or Y₁—Rₐ; wherein:
Yₜ is —C(O)—, —C(O)O—, —C(O)NR_{b}—, —S(O)ₓ—, —S(O)ₓO—, or —S(O)ₓNR_{b}—; wherein:
x is 0, 1, or 2;
R_{b} and R_{b′} are each independently hydrogen, alkyl_{(C≤12)}, substituted alkyl_{(C≤12)}, acyl_{(C≤12)}, substituted acyl_{(C≤12)}, or a monovalent amino protecting group; and
Rₐ is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
R₃ is hydrogen, alkyl_{(C≤12)}, aryl_{(C≤12)}, aralkyl_{(C≤12)}, heteroaryl_{(C≤12)}, heterocycloalkyl_{(C≤12)}, or a substituted version of any of these groups; or is the side chain of one of the 20 canonical amino acids;
R₄ and R₆ are each independently selected from hydrogen, alkyl_{(C≤12)}, substituted alkyl_{(C≤12)}, acyl_{(C≤12)}, substituted acyl_{(C≤12)}, or a monovalent amino protecting group; and
R₅ is Y₂—R_{c}; wherein:
Y₂ is —NR_{d}—, —S—, —O—, —C(O)—, —OC(O)—, —C(O)O—, —NR_{d}C(O)—, —C(O)NR_{d}—, —OC(O)O—, —OC(O)NR_{d}—, —NR_{d}C(O)O—, —NR_{d}C(O)NR_{d}—, —S(O)—, —OS(O)—, —S(O)_{y}O—, —NR_{b}S(O)_{y}—, —S(O)ₓNR_{d}—, —OS(O)_{y}O—, —OS(O)_{y}NR_{d}—, —NR_{d}S(O)O—, or —NR_{d}S(O)_{y}NR_{d′}—; wherein:
y is 0, 1, or 2;
R_{d} and R_{d′} are each independently hydrogen, alkyl_{(C≤12)}, substituted alkyl_{(C≤12)}, acyl_{(C≤12)}, substituted acyl_{(C≤12)}, or a monovalent amino protecting group; and
R_{c} is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
or a pharmaceutically acceptable salt thereof.

3. The compound of either claim 1 or claim 2, further defined as: 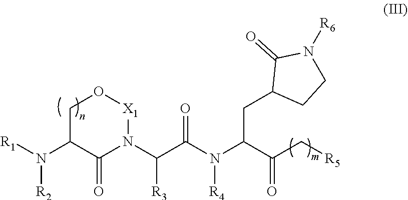
wherein:
X₁ is cycloalkanediyl_{(C≤12)}, arenediyl_{(C≤12)}, heteroarenediyl_{(C≤12)}, heterocycloalkanediyl_{(C≤12)}, or a substituted version of any of these groups;
m and n are each independently 0, 1, 2, or 3;
R₁ and R₂ are each independently selected from hydrogen, alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; or a monovalent amino protecting group; or R₁ and R₂ are a divalent amino protecting group; or Y₁—Rₐ; wherein:
Y₁ is —C(O)—, —C(O)O—, —C(O)NR_{b}—, —S(O)ₓ—, —S(O)ₓO—, or —S(O)ₓNR_{b}—; wherein:
x is 0, 1, or 2;
R_{b} and R_{b′} are each independently hydrogen, alkyl_{(C≤12)}, substituted alkyl_{(C≤12)}, acyl_{(C≤12)}, substituted acyl_{(C≤12)}, or a monovalent amino protecting group; and
Rₐ is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
R₃ is hydrogen, alkyl_{(C≤12)}, aryl_{(C≤12)}, aralkyl_{(C≤12)}, heteroaryl_{(C≤12)}, heterocycloalkyl_{(C≤12)}, or a substituted version of any of these groups; or is the side chain of one of the 20 canonical amino acids;
R_{d} and R_{d′} are each independently selected from hydrogen, alkyl_{(C≤12)}, substituted alkyl_{(C≤12)}, acyl_{(C≤12)}, substituted acyl_{(C≤12)}, or a monovalent amino protecting group; and
R₅ is Y₂—R_{c}; wherein:
Y₂ is —NR_{d}—, —S—, —O—, —C(O)—, —OC(O)—, —C(O)O—, —NR_{d}C(O)—, —C(O)NR_{d}—, —OC(O)O—, —OC(O)NR_{d}—, —NR_{d}C(O)O—, —NR_{d}C(O)NR_{d}—, —S(O)_{y}—, —OS(O)_{y}—, —S(O)_{y}O—, —NR_{b}S(O)_{y}—, —S(O)_{y}NR_{d}—, —OS(O)_{y}O—, —OS(O)_{y}NR_{d}—, —NR_{d}S(O)_{y}O—, or —NR_{d}S(O)_{y}NR_{d′}—; wherein:
y is 0, 1, or 2;
R_{d} and R_{d′} are each independently hydrogen, alkyl_{(C≤12)}, substituted alkyl_{(C≤12)}, acyl_{(C≤12)}, substituted acyl_{(C≤12)}, or a monovalent amino protecting group; and
R_{c} is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
or a pharmaceutically acceptable salt thereof.

4. The compound according to any one of claims 1-3 further defined as: 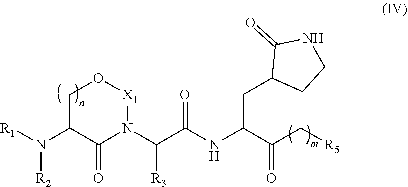
wherein:
X₁ is cycloalkanediyl_{(C≤12)}, arenediyl_{(C≤12)}, heteroarenediyl_{(C≤12)}, heterocycloalkanediyl_{(C≤12)}, or a substituted version of any of these groups;
m and n are each independently 0, 1, 2, or 3;
R₁ and R₂ are each independently selected from hydrogen, alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; or a monovalent amino protecting group; or R₁ and R₂ are a divalent amino protecting group; or Y₁—Rₐ; wherein:
Yₜ is —C(O)—, —C(O)O—, —C(O)NR_{b}—, —S(O)ₓ—, —S(O)ₓO—, or —S(O)ₓNR_{b}—; wherein:
x is 0, 1, or 2;
R_{b} and R_{b′} are each independently hydrogen, alkyl_{(C≤12)}, substituted alkyl_{(C≤12)}, acyl_{(C≤12)}, substituted acyl_{(C≤12)}, or a monovalent amino protecting group; and
Rₐ is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
R₃ is hydrogen, alkyl_{(C≤12)}, aryl_{(C≤12)}, aralkyl_{(C≤12)}, heteroaryl_{(C≤12)}, heterocycloalkyl_{(C≤12)}, or a substituted version of any of these groups; or is the side chain of one of the 20 canonical amino acids; and
R₅ is Y₂—R_{c}; wherein:
Y₂ is —NR_{d}—, —S—, —O—, —C(O)—, —OC(O)—, —C(O)O—, —NR_{d}C(O)—, —C(O)NR_{d}—, —OC(O)O—, —OC(O)NR_{d}—, —NR_{d}C(O)O—, —NR_{d}C(O)NR_{d′}—, —S(O)_{y}—, —OS(O)_{y}—, —S(O)_{y}O—, —NR_{b}S(O)_{y}—, —S(O)_{y}NR_{d}—, —OS(O)_{y}O—, —OS(O)_{y}NR_{d}—, —NR_{d}S(O)_{y}O—, or —NR_{d}S(O)_{y}NR_{d′}—; wherein:
y is 0, 1, or 2;
R_{d} and R_{d′} are each independently hydrogen, alkyl_{(C≤12)}, substituted alkyl_{(C≤12)}, acyl_{(C≤12)}, substituted acyl_{(C≤12)}, or a monovalent amino protecting group; and
R_{c} is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
or a pharmaceutically acceptable salt thereof.

5. The compound according to any one of claims 1-4 further defined as: 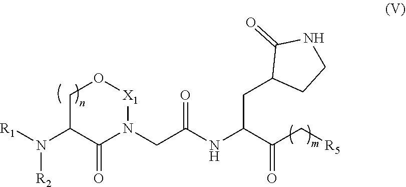
wherein:
X₁ is cycloalkanediyl_{(C≤12)}, arenediyl_{(C≤12)}, heteroarenediyl_{(C≤12)}, heterocycloalkanediyl_{(C≤12)}, or a substituted version of any of these groups;
m and n are each independently 0, 1, 2, or 3;
R₁ and R₂ are each independently selected from hydrogen, alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; or a monovalent amino protecting group; or R₁ and R₂ are a divalent amino protecting group; or Y₁—Rₐ; wherein:
Y₁ is —C(O)—, —C(O)O—, —C(O)NR_{b}—, —S(O)ₓ—, —S(O)ₓO—, or —S(O)ₓNR_{b}—; wherein:
x is 0, 1, or 2;
R_{b} and R_{b′} are each independently hydrogen, alkyl_{(C≤12)}, substituted alkyl_{(C≤12)}, acyl_{(C≤12)}, substituted acyl_{(C≤12)}, or a monovalent amino protecting group; and
Rₐ is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; and
R₅ is Y₂—R_{c}; wherein:
Y₂ is —NR_{d}—, —S—, —O—, —C(O)—, —OC(O)—, —C(O)O—, —NR_{d}C(O)—, —C(O)NR_{d}—, —OC(O)O—, —OC(O)NR_{d}—, —NR_{d}C(O)O—, —NR_{d}C(O)NR_{d′}—, —S(O)_{y}—, —OS(O)_{y}—, —S(O)_{y}O—, —NR_{b}S(O)_{y}—, —S(O)ₓNR_{d}—, —OS(O)_{y}O—, —OS(O)_{y}NR_{d}—, —NR_{d}S(O)_{y}O—, or —NR_{d}S(O)_{y}NR_{d′}—; wherein:
y is 0, 1, or 2;
R_{d} and R_{d′} are each independently hydrogen, alkyl_{(C≤12)}, substituted alkyl_{(C≤12)}, acyl_{(C≤12)}, substituted acyl_{(C≤12)}, or a monovalent amino protecting group; and
R_{c} is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
or a pharmaceutically acceptable salt thereof.

6. The compound according to any one of claims 1-5 further defined as: 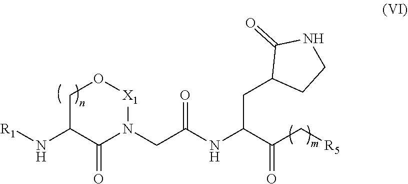
wherein:
X₁ is cycloalkanediyl_{(C≤12)}, arenediyl_{(C≤12)}, heteroarenediyl_{(C≤12)}, heterocycloalkanediyl_{(C≤12)}, or a substituted version of any of these groups;
m or n are each independently 0, 1, 2, or 3;
R₁ is hydrogen, alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; or a monovalent amino protecting group; or Y₁—Rₐ; wherein:
Y₁ is —C(O)—, —C(O)O—, —C(O)NR_{b}—, —S(O)ₓ—, —S(O)ₓO—, or —S(O)ₓNR_{b}—; wherein:
x is 0, 1, or 2;
R_{b} and R_{b′} are each independently hydrogen, alkyl_{(C≤12)}, substituted alkyl_{(C≤12)}, acyl_{(C≤12)}, substituted acyl_{(C≤12)}, or a monovalent amino protecting group; and
Rₐ is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; and
R₅ is Y₂—R_{c}; wherein:
Y₂ is —NR_{d}—, —S—, —O—, —C(O)—, —OC(O)—, —C(O)O—, —NR_{d}C(O)—, —C(O)NR_{d}—, —OC(O)O—, —OC(O)NR_{d}—, —NR_{d}C(O)O—, —NR_{d}C(O)NR_{d′}—, —S(O)_{y}—, —OS(O)—, —S(O)_{y}O—, —NR_{b}S(O)_{y}—, —S(O)_{y}NR_{d}—, —OS(O)_{y}O—, —OS(O)_{y}NR_{d}—, —NR_{d}S(O)_{y}O—, or —NR_{d}S(O)_{y}NR_{d′}—; wherein:
y is 0, 1, or 2;
R_{d} and R_{d′} are each independently hydrogen, alkyl_{(C≤12)}), substituted alkyl_{(C≤12)}, acyl_{(C≤12)}, substituted acyl_{(C≤12)}, or a monovalent amino protecting group; and
R_{c} is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
or a pharmaceutically acceptable salt thereof.

7. The compound according to any one of claims 1-6 further defined as: 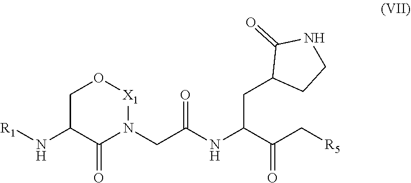
wherein:
X₁ is cycloalkanediyl_{(C≤12)}, arenediyl_{(C≤12)}, heteroarenediyl_{(C≤12)}, heterocycloalkanediyl_{(C≤12)}, or a substituted version of any of these groups;
R₁ is hydrogen, alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; or a monovalent amino protecting group; or Y₁—Rₐ; wherein:
Y₁ is —C(O)—, —C(O)O—, —C(O)NR_{b}—, —S(O)ₓ—, —S(O)ₓO—, or —S(O)ₓNR_{b}—; wherein:
x is 0, 1, or 2;
R_{b} and R_{b′} are each independently hydrogen, alkyl_{(C≤12)}, substituted alkyl_{(C≤12)}, acyl_{(C≤12)}, substituted acyl_{(C≤12)}, or a monovalent amino protecting group; and
Rₐ is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups; and
R₅ is Y₂—R_{c}; wherein:
Y₂ is —NR_{d}—, —S—, —O—, —C(O)—, —OC(O)—, —C(O)O—, —NR_{d}C(O)—, —C(O)NR_{d}—, —OC(O)O—, —OC(O)NR_{d}—, —NR_{d}C(O)O—, —NR_{d}C(O)NR_{d}—, —S(O)_{y}—, —OS(O)_{y}—, —S(O)O—, —NR_{b}S(O)_{y}—, —S(O)_{y}NR_{d}—, —OS(O)O—, —OS(O)_{y}NR_{d}—, —NR_{d}S(O)_{y}O—, or —NR_{d}S(O)_{y}NR_{d′}—; wherein:
y is 0, 1, or 2;
R_{d} and R_{d′} are each independently hydrogen, alkyl_{(C≤12)}, substituted alkyl_{(C≤12)}, acyl_{(C≤12)}, substituted acyl_{(C≤12)}, or a monovalent amino protecting group; and
R_{c} is alkyl_{(C≤12)}, alkenyl_{(C≤12)}, alkynyl_{(C≤12)}, aryl_{(C≤12)}, aralkyl_{(C≤12)}, heteroaryl_{(C≤12)}, heterocycloalkyl_{(C≤12)}, acyl_{(C≤12)}, or a substituted version of any of these groups;
or a pharmaceutically acceptable salt thereof.

8. The compound according to any one of claims 3-7, wherein X₁ is arenediyl_{(C≤12)} or substituted arenediyl_{(C≤12)}.

9. The compound of claim 8, wherein X₁ is arenediyl_{(C≤12)}.

10. The compound of claim 9, wherein X₁ is benzenediyl.

11. The compound according to any one of claims 1-10, wherein R₁ is Y₁—Rₐ.

12. The compound of claim 11, wherein Y₁ is —C(O)—, —C(O)O—, or —C(O)NR_{b}—.

13. The compound of claim 12, wherein Y₁ is —C(O)O—.

14. The compound according to any one of claims 11-13, wherein Rₐ is aralkyl_{(C≤12)} or substituted aralkyl_{(C≤12)}.

15. The compound of claim 14, wherein Rₐ is aralkyl_{(C≤12)}.

16. The compound of claim 15, wherein Rₐ is benzyl.

17. The compound of either claim 11 or claim 12, wherein Y₁ is —C(O)—.

18. The compound according to any one of claims 11, 12, or 17, wherein Rₐ is aryl_{(C≤12)} or substituted aryl_{(C≤12)}.

19. The compound of claim 18, wherein Rₐ is aryl_{(C≤12)}.

20. The compound of claim 19, wherein Rₐ is phenyl.

21. The compound according to any one of claims 11, 12, or 17, wherein Rₐ is heteroaryl_{(C≤12)} or substituted heteroaryl_{(C≤12)}.

22. The compound of claim 21, wherein Rₐ is heteroaryl_{(C≤12)}.

23. The compound of claim 22, wherein Rₐ is quinolyl.

24. The compound of claim 23, wherein Rₐ is 2-quinolyl.

25. The compound according to any one of claims 1-24, wherein Y₂ is —OC(O)— or —C(O)O—.

26. The compound of claim 25, wherein Y₂ is —OC(O)—.

27. The compound according to any one of claims 1-26, wherein R_{c} is aryl_{(C≤12)} or substituted aryl_{(C≤12)}.

28. The compound of claim 27, wherein R_{c} is substituted aryl_{(C≤12)}.

29. The compound of claim 28, wherein R_{c} is haloaryl_{(C≤12)}.

30. The compound of claim 29, wherein R_{c} is 2,6-dichlorophenyl.

31. The compound according to any one of claims 1-6 and 8-30, wherein m is 0, 1, or 2.

32. The compound of claim 31, wherein m is 0 or 1.

33. The compound of claim 32, wherein m is 1 or 2.

34. The compound according to any one of claims 31-33, wherein m is 1.

35. The compound according to any one of claims 1-6 and 8-34, wherein n is 0, 1, or 2.

36. The compound of claim 35, wherein n is 0 or 1.

37. The compound of claim 35, wherein n is 1 or 2.

38. The compound according to any one of claims 35-37, wherein n is 1.

39. The compound according to any one of claims 1-5 and 8-38, wherein R₂ is hydrogen.

40. The compound according to any one of claims 1, 4, and 8-39, wherein R₃ is hydrogen.

41. The compound according to any one of claims 1 and 8-40, wherein R₄ is hydrogen.

42. The compound according to any one of claims 1 and 8-41, wherein R₆ is hydrogen.

43. The compound according to any one of claims 1-42, wherein the compound is further defined as: 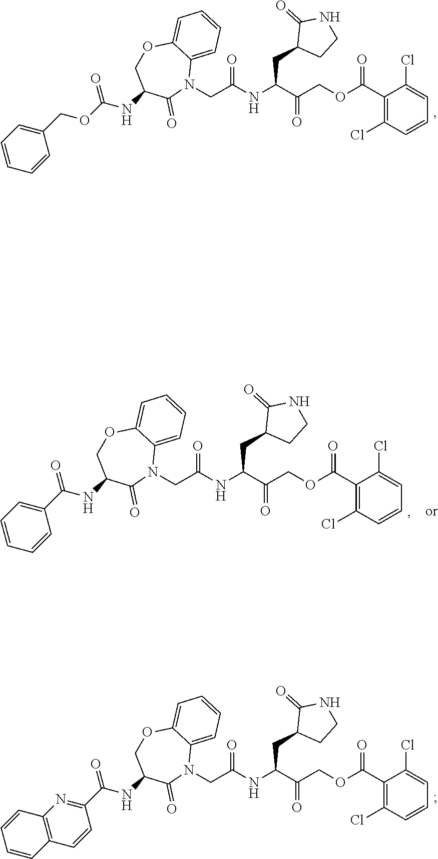 [SMILES: O=C(CN1C(=O)[C@@H](NC(=O)OCc2ccccc2)COc2ccccc21)N[C@@H](C[C@@H]1CCNC1=O)C(=O)COC(=O)c1c(Cl)cccc1Cl ; O=C(CN1C(=O)[C@@H](NC(=O)c2ccccc2)COc2ccccc21)N[C@@H](C[C@@H]1CCNC1=O)C(=O)COC(=O)c1c(Cl)cccc1Cl ; O=C(CN1C(=O)[C@@H](NC(=O)c2ccc3ccccc3n2)COc2ccccc21)N[C@@H](C[C@@H]1CCNC1=O)C(=O)COC(=O)c1c(Cl)cccc1Cl]
or a pharmaceutically acceptable salt thereof.

44. A pharmaceutical composition comprising:
(A) a compound according to any one of claims 1-43; and
(B) an excipient.

45. The pharmaceutical composition of claim 44, wherein the pharmaceutical composition is formulated for administration systemically.

46. The pharmaceutical composition of either claim 44 or claim 45, wherein the pharmaceutical composition is formulated as a unit dose.

47. A method of treating a disease or disorder in a patient comprising administering to the patient in need thereof a therapeutically effective dose of a compound or pharmaceutical composition according to any one of claims 1-46.

48. The method of claim 47, wherein the disease or disorder is a viral infection.

49. The method of claim 48, wherein the viral infection is the infection of a coronavirus.

50. The method of claim 49, wherein the coronavirus is SARS-CoV-2 or a variant thereof.

51. The method according to any one of claims 47-50, wherein the patient is a mammal.

52. The method of claim 51, wherein the mammal is human.

53. The method according to any one of claims 47-52, wherein the patient has been diagnosed with the infection.

54. The method according to any one of claims 47-52, wherein the patient has not been diagnosed with the infection.

55. The method according to any one of claims 47-54, wherein the compound is administered with a second therapeutic agent.

56. The method of claim 55, wherein the second therapeutic agent is molnupiravir, paxlovid, or remdesivir.

57. The method of claim 56, wherein the second therapeutic agent is remdesivir.

58. The method according to any one of claims 55-57, wherein the method comprises administering less than a therapeutically effective dose of remdesivir.

59. The method according to any one of claims 55-58, wherein the method comprises administering less than a therapeutically effective dose of the compound when the compound is administered alone.

60. The method of either claim 58 or claim 59, wherein the method comprises administering both remdesivir and the compound in less than a therapeutically effective dose.

61. The method according to any one of claims 47-60, wherein the compound is administered for 1 day to 20 days.

62. The method of claim 61, wherein the compound is administered for 3 days to 5 days.

63. The method according to any one of claims 47-60, wherein the compound is administered once.

64. The method according to any one of claims 47-62, wherein the compound is administered two or more times.
